(19) United States
(12) Patent Application Publication
(10) Pub. No.: US 2023/0109275 A1
(43) Pub. Date: Apr. 6, 2023

(54) TRBC BETA ANTIBODY CONJUGATE

(71) Applicant: AUTOLUS LIMITED, London (GB)

(72) Inventors: Shimobi Onuoha, London (GB); Mathieu Ferrari, London (GB); Zulaikha Akbar, London (GB); Reyisa Bughda, London (GB); Martin Pulé, London (GB)

(21) Appl. No.: 17/913,649

(22) PCT Filed: Mar. 24, 2021
(86) PCT No.: PCT/GB2021/050719

§ 371 Date: Sep. 22, 2022

(30) Foreign Application Priority Data
Mar. 24, 2020 (GB) 2004263.6

(51) Int. Cl. A61K 47/68; C07K 16/28; A61P 35/02

(52) U.S. Cl. CPC A61K 47/6849; C07K 16/2809; A61K 47/6817; A61P 35/02; C07K 2317/92; C07K 2317/56

(57) ABSTRACT

The present disclosure provides an antibody conjugate that binds specifically to TCR beta constant region (TRBC), wherein the antibody has a fast dissociation rate constant (k_{d}). It further provides medical uses and methods of personalised medicine that exploit the products of the invention.

### FIELD OF THE INVENTION

The present invention relates to an antibody conjugate that binds specifically to TCR beta constant region (TRBC), wherein the antibody has a fast dissociation rate constant (kd).

### BACKGROUND TO THE INVENTION

Lymphoid malignancies can largely be divided into those which are derived from either T-cells or B-cells. T-cell malignancies are a clinically and biologically heterogeneous group of disorders, together comprising 10-20% of non-Hodgkin's lymphomas and 20% of acute leukaemias. The most commonly identified histological subtypes are peripheral T-cell lymphoma, not otherwise specified (PTCL-NOS); angio-immunoblastic T-cell lymphoma (AITL) and anaplastic large cell lymphoma (ALCL). Of all acute Lymphoblastic Leukaemias (ALL), some 20% are of a T-cell phenotype.

These conditions typically behave aggressively, compared for instance with B-cell malignancies, with estimated 5-year survival of only 30%. In the case of T-cell lymphoma, they are associated with a high proportion of patients presenting with disseminated disease, unfavourable International Prognostic Indicator (IPI) score and prevalence of extra-nodal disease. Chemotherapy alone is not usually effective and less than 30% of patients are cured with current treatments.

Further, unlike in B-cell malignancies, where immunotherapies such as the anti-CD20 monoclonal antibody rituximab have dramatically improved outcomes, there is currently no equivalently effective, minimally toxic immunotherapeutic available for the treatment of T-cell malignancies. An important difficulty in the development of immunotherapy for T-cell disorders is the considerable overlap in marker expression of clonal and normal T-cells, with no single antigen clearly able to identify clonal (malignant) cells.

A targeting strategy based on the mutually exclusive expression of T cell receptor beta-chain constant domains 1 and 2 (TRBC1 and TRBC2) has been reported (WO2015/132598; Maciocia et al., 2017, Nat Med 23:1416-23). Moreover, it has been demonstrated that CARs targeting either TRBC1 or TRBC2 offer the ability to treat T-cell lymphomas, while potentially providing an acceptable toxicity profile (Maciocia et al., 2017, Nat Med 23:1416-23; WO2015/132598).

However, diseases such as Human T-cell leukaemia virus, type 1 (HTLV-1) associated leukaemias and lymphomas may not be well suited to cell therapy despite having the potential to be treated with TRBC1/TRBC2 targeting drugs. This is because cell-based therapies may be prone to T cell-mediated fratricide.

Therefore, there is a need in the art to provide alternative targeting agents that overcome the potential disadvantages of cell-based therapies in the treatment of T cell lymphomas and leukaemias.

Antibody drug conjugates (ADCs) offer another immunotherapeutical modality which can be used for targeting the T cell lymphomas. ADCs offer further advantages over cell-based therapies in that they are not prone to T cell mediated fratricide. Furthermore, ADCs may offer improved management of side effects.

The efficiency of the ADC strategy largely depends on the internalization of the cytotoxic conjugates into cancer cells. ADCs based on TRBC specific antibodies have been described previously (WO2015/132598) but their internalisation properties are unknown.

Important features of the antibody suitable for ADC are that the antibody specifically binds to TRBC as well as that it has high internalisation ability. The internalisation ability of the antibody depends on the properties of both the target antigen and the antibody. It is difficult to predict an antigen-binding site suitable for internalisation from the molecular structure of a target or easily to predict an antibody having high internalisation ability from binding strength, physical properties, and the like of the antibody. Hence, an important challenge in developing ADC having high efficacy is obtaining an antibody having high internalisation ability against the target antigen

There is therefore a need in the art for TRBC-specific ADCs with optimal internalisation properties.

### SUMMARY OF ASPECTS OF THE INVENTION

The present inventors have investigated the ADC therapeutic strategy for T cell lymphomas and leukaemias using a number of TRBC-specific antibodies of varying affinities with the aim of characterising their internalisation properties upon binding to T cells. The results obtained served to identify binding affinities which confers the specific antibodies with good internalisation properties. Unexpectedly, the present inventors determined that a fast dissociation rate constant is important in order to achieve good internalisation of the antibodies. These results are in contrast with the view that is commonly accepted in the art, i.e. that high affinity antibodies, and therefore having a slow dissociation rate constant, display high internalisation ability.

Thus, in a first aspect, the present invention provides an antibody conjugate that binds specifically to TCR beta constant region (TRBC), wherein the antibody has a dissociation rate constant (k_{d}) in the range of 0.001 s⁻¹ to 0.3 s⁻¹.

The antibody may have a k_{d} in the range of 0.002 s⁻¹ to 0.1 s⁻¹.

The antibody may be conjugated to a chemotherapeutic entity, a radionuclide or a detection entity.

The chemotherapeutic entity may be a tubulin inhibitor.

The tubulin inhibitor may be MMAE.

The antibody conjugate may have an increased internalisation upon binding to a target cell compared to that of a reference antibody having a VH domain with the sequence shown in SEQ ID NO: 1 and a VL domain with the sequence shown in SEQ ID NO: 2.

The antibody may bind specifically to TRBC1.

The TRBC1-specific antibody may comprise one of the following mutations compared to a reference antibody having a VH domain with the sequence shown in SEQ ID NO: 1 and a VL domain with the sequence shown in SEQ ID NO: 2:
G106A in the VH domain;
Y32F in the VH domain;
G31S in the VH domain;
G26P and T28K in the VH domain;
Y102M in the VH domain.

The TRBC1-specific antibody may comprise the following mutation compared to a reference antibody having a VH domain with the sequence shown in SEQ ID NO: 1 and a VL domain with the sequence shown in SEQ ID NO: 2:
G106A in the VH domain.

The antibody may bind specifically to TRBC2.

The TRBC2-specific antibody may comprise one of the following mutation combinations compared to a reference antibody having a VH domain with the sequence shown in SEQ ID NO: 1 and a VL domain with the sequence shown in SEQ ID NO: 2:
T28K, Y32F, A100N, Y102L, and N103M in the VH domain, and VL N35R in the VL domain;
T28K, Y32F, A100N in the VH domain; or
T28R, Y32F, A100N in the VH domain.

The TRBC2-specific antibody may comprise the following mutations compared to a reference antibody having a VH domain with the sequence shown in SEQ ID NO: 1 and a VL domain with the sequence shown in SEQ ID NO: 2:
T28K, Y32F, A100N, Y102L, and N103M in the VH domain, and VL N35R in the VL domain.

In a second aspect, the present invention provides an antibody conjugate according to the first aspect of the invention for use in the treatment of a T cell lymphoma or leukaemia.

The treatment of a T cell lymphoma or leukaemia in a subject may comprise the step of administrating the antibody conjugate to the subject, to cause selective depletion of the malignant T-cells, together with normal T-cells expressing the same TRBC as the malignant T-cells, but not to cause depletion of normal T-cells expressing the TRBC not expressed by the malignant T-cells.

The method may further comprise the step of investigating the TCR beta constant region (TCRB) of a malignant T cell from the subject to determine whether it expresses TRBC1 or TRBC2.

The T-cell lymphoma or leukaemia may be selected from peripheral T-cell lymphoma, not otherwise specified (PTCL-NOS); angio-immunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, T cell prolymphocytic leukaemia and T-cell acute lymphoblastic leukaemia.

In a third aspect, the present invention provides an antibody conjugate according to embodiments of the first aspect of the invention for use in a method for targeting the delivery of a chemotherapeutic drug to a cell which expresses TRBC in a subject.

In a fourth aspect, the present invention provides a pharmaceutical composition which comprises an antibody conjugate according to the first aspect of the invention and a pharmaceutically acceptable carrier, diluent, excipient or adjuvant.

In a fifth aspect, the present invention provides a method for selecting a suitable therapy to treat a subject suffering from T-cell lymphoma or leukaemia which comprises:
i) determining whether a malignant T cell in a sample isolated from the subject expresses TRBC1 or TRBC2; and
ii) selecting an antibody conjugate for use according to the second aspect of the invention based on the TRBC1 or TRBC2 expression of said malignant T cell.

In a sixth aspect, the present invention provides a method for selecting a subject suffering from T-cell lymphoma or leukaemia to receive a therapy comprising an antibody conjugate for use according to the second aspect of the invention, which comprises:
i) determining whether a malignant T cell in a sample isolated from the subject expresses TRBC1 or TRBC2; and
ii) selecting said subject to receive a therapy based on an antibody conjugate for use according to the second aspect of the invention based on the TRBC1 or TRBC2 expression of said malignant T cell.

### DESCRIPTION OF THE FIGURES

FIG. 1. A diagram of the αβ T-cell Receptor/CD3 Complex. The T-cell receptor is formed from 6 different protein chains which must assemble in the endoplasmic reticulum to be expressed on the cell surface. The four proteins of the CD3 complex (CD3ζ, CD3γ, CD3ε and CD3δ) sheath the T-cell Receptor (TCR). This TCR imbues the complex with specificity of a particular antigen and is composed of two chains: TCRα and TCRβ. Each TCR chain has a variable component distal to the membrane and a constant component proximal to the membrane. Nearly all T-cell lymphomas and many T-cell leukaemias express the TCR/CD3 complex.

FIG. 2: The segregation of T-cell Receptor β-constant region (TRBC)-1 and TRBC2 during T-cell receptor rearrangement. Each TCR beta chain is formed from genomic recombination of a particular beta variable (V), diversity (D), joining (J) and constant (TRBC) regions. The human genome contains two very similar and functionally equivalent TRBC loci known as TRBC1 and TRBC2. During TCR gene re-arrangement, a J-region recombines with either TRBC1 or TRBC2. This rearrangement is permanent. T-cells express many copies of a single TCR on their surface, hence each T-cell will express a TCR whose β-chain constant region is coded for by either TRBC1 or TRBC2.

FIG. 3. Structural interface between TCR Beta and Fab fragment of TRBC1 specific antibody Jovi-1.

FIG. 4. Internalisation profile of pHrodo red conjugated Jovi-1, KFN and anti-HEL antibody in HPB-ALL TRBC1, HPB-ALL TRBC2 and HPB-ALL KO cells.

FIG. 5. Surface plasmon resonance experiments carried out on a variety of mutated antibody variants to ascertain affinity, association and dissociation rates

FIG. 6. Analysis of binding kinetics of mutated antibody variants. A number of binders with highly similar association rates but with different dissociation rates were identified.

FIG. 7. Internalisation profile of pHrodo green conjugated mutants of aTRBC1 in HPB-ALL TRBC1 and KO cells at 9 h. Clones were selected with increasing dissociation rates for TRBC1. Flow cytometry shows improved internalisation of binders with faster dissociation rates up to Mut13 before internalisation is affected by faster off-rates. Antibody mutants are described in Tables 1 and 2.

FIG. 8. Internalisation of optimal affinity TRBC1 and TRBC 2 antibodies on HPB-ALL TRBC1+, HPB-ALL TRBC2+, and HPB-ALL TCR KO cells. Antibody mutants are described in Tables 1 and 2.

FIG. 9. A. Schematic representation of anti-TCR PAB-MMAE conjugation via MC-Valine-Citrulline linker. B. Conjugation of Mut11 and Mut15 antibodies to monomethyl auristatin E (MMAE) did not impair ability for antibodies to be internalised. MFI: mean fluorescence intensity. C. Cytotoxicity assay on HPB-ALL TRBC1+, HPB-ALL TRBC2+ and HPB-ALL TCR KO with MMAE conjugated Mut11, Mut15, and anti-HEL. ****P<0.0001 by comparison of fit versus HPB-ALL TCR KO. Antibody mutants are described in Tables 1 and 2.

FIG. 10. Diagram of the structure of TRBC1 and TRBC2 specific chimeric antigen receptors (CARs).

### DETAILED DESCRIPTION OF THE INVENTION

The present invention provides antibody conjugates that bind specifically to TCR beta constant region (TRBC), which have good internalisation properties upon binding the target cell.

1. TCR β Constant Region (TRBC)

The T-cell receptor (TCR) is expressed on the surface of T lymphocytes and is responsible for recognizing antigens bound to major histocompatibility complex (MHC) molecules. When the TCR engages with antigenic peptide and MHC (peptide/MHC), the T lymphocyte is activated through a series of biochemical events mediated by associated enzymes, co-receptors, specialized adaptor molecules, and activated or released transcription factors.

The TCR is a disulfide-linked membrane-anchored heterodimer normally consisting of the highly variable alpha (a) and beta (β) chains expressed as part of a complex with the invariant CD3 chain molecules. T-cells expressing this receptor are referred to as α:β (or αβ) T-cells (˜95% total T-cells). A minority of T-cells express an alternate receptor, formed by variable gamma (γ) and delta (δ) chains, and are referred to as γδ T-cells (˜5% total T cells).

Each α and β chain is composed of two extracellular domains: Variable (V) region and a Constant (C) region, both of Immunoglobulin superfamily (IgSF) domain forming antiparallel β-sheets. The constant region is proximal to the cell membrane, followed by a transmembrane region and a short cytoplasmic tail, while the variable region binds to the peptide/MHC complex. The constant region of the TCR consists of short connecting sequences in which a cysteine residue forms disulfide bonds, which forms a link between the two chains.

The variable domains of both the TCR α-chain and β-chain have three hypervariable or complementarity determining regions (CDRs). The variable region of the β-chain also has an additional area of hypervariability (HV4), however, this does not normally contact antigen and is therefore not considered a CDR.

The TCR also comprises up to five invariant chains γ,δ,ε (collectively termed CD3) and ζ The CD3 and ζ subunits mediate TCR signalling through specific cytoplasmic domains which interact with second-messenger and adapter molecules following the recognition of the antigen by αβ or γδ. Cell-surface expression of the TCR complex is preceded by the pair-wise assembly of subunits in which both the transmembrane and extracellular domains of TCR α and β and CD3 γ and δ play a role

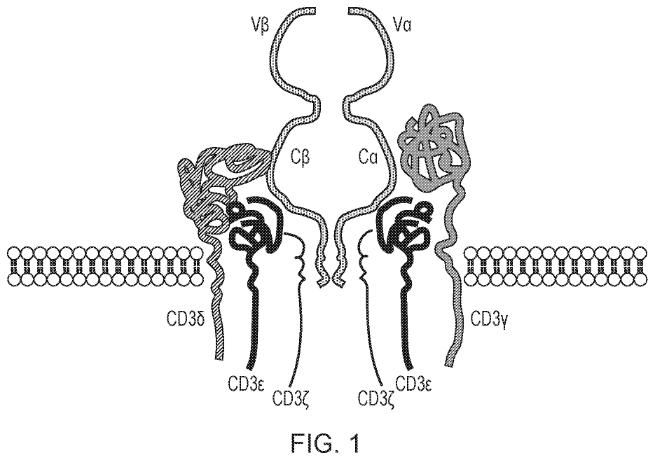

TCRs are therefore commonly composed of the CD3 complex and the TCR α and β chains, which are in turn composed of variable and constant regions (FIG. 1).

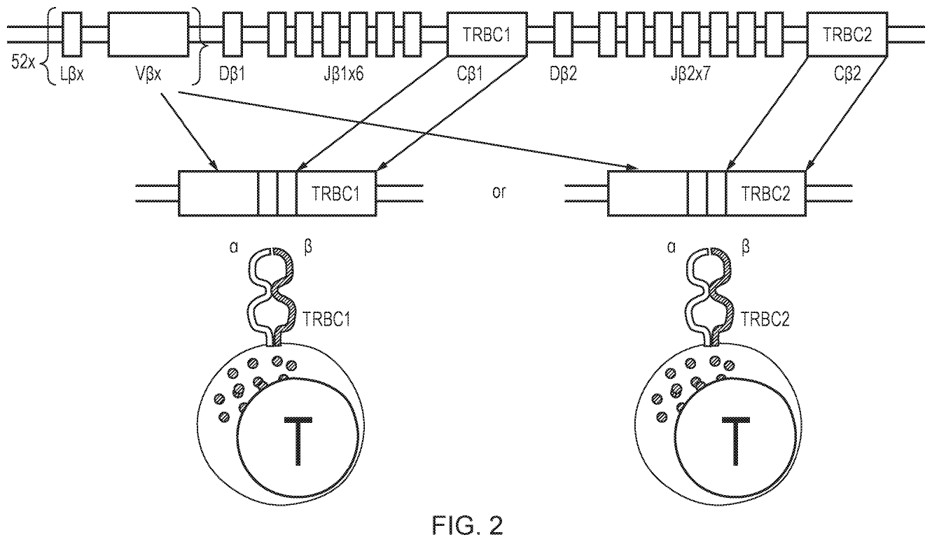

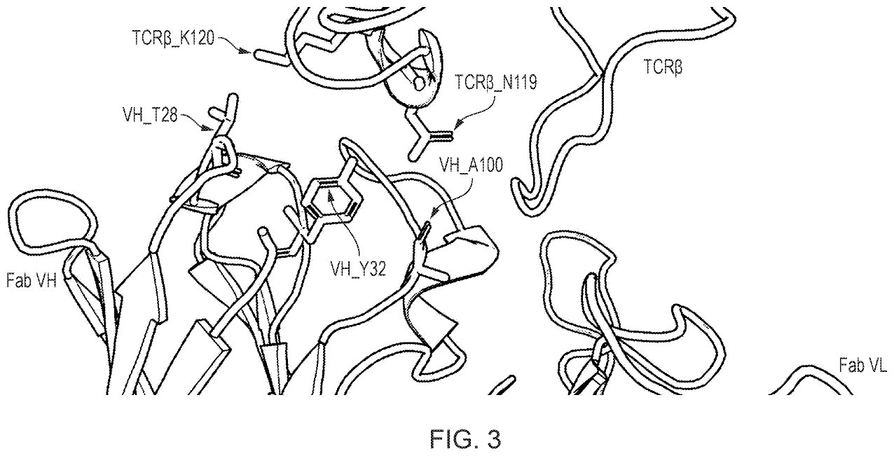

The locus (Chr7:q34) which supplies the TCR β-constant region (TRBC) has duplicated in evolutionary history to produce two almost identical and functionally equivalent genes: TRBC1 and TRBC2 (FIG. 2). Each TCR will comprise, in a mutually exclusive fashion, either TRBC1 or TRBC2 and as such, each αβ T-cell will express either TRBC1 or TRBC2, in a mutually exclusive manner.

The present inventors have previously determined that, despite the similarity between the sequence of the TRBC1 and TRBC2, it is possible to discriminate between them. The inventors have also previously determined that amino acid sequences of TRBC1 and TRBC2 can be discriminated whilst in situ on the surface of a cell, for example a T-cell (WO2015132598). Additionally, numerous antibodies with specificity to either TRBC1 or TRBC2 have been generated (WO2015132598).

2. Antibody Conjugate

In a first aspect, the present invention provides an antibody conjugate that binds specifically to TCR beta constant region (TRBC), hereinafter “the antibody conjugate of the invention”, wherein the antibody has a dissociation rate constant (k_{d}) in the range of 0.001 s⁻¹ to 0.5 s⁻¹.

The term “antibody conjugate”, as used herein, refers to a compound comprising an antibody attached to a therapeutic entity or payload via chemical linkers. Upon binding to the target antigen on the surface of a cell, the antibody conjugate is internalised and trafficked to the lysosome where the payload is released by either proteolysis of a cleavable linker (e.g., by cathepsin B found in the lysosome) or by proteolytic degradation of the antibody, if attached to the payload via a non-cleavable linker.

2.1. Antibody

The antibody conjugate of the invention comprises an antibody that binds specifically to TCR beta constant region (TRBC).

The term “antibody”, as used herein, refers to a polypeptide having an antigen binding site which comprises at least one complementarity determining region or CDR. The antibody may comprise 3 CDRs and have an antigen binding site which is equivalent to that of a single domain antibody (dAb), heavy chain antibody (VHH) or a nanobody (see FIG. 3b). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen.

A full-length antibody or immunoglobulin typically consists of four polypeptides: two identical copies of a heavy (H) chain polypeptide and two identical copies of a light (L) chain polypeptide. Each of the heavy chains contains one N terminal variable (VH) region and three C-terminal constant (CH₁, CH₂ and CH₃) regions, and each light chain contains one N-terminal variable (VL) region and one C-terminal constant (CL) region. The variable regions of each pair of light and heavy chains form the antigen binding site of an antibody. They are characterised by the same general structure constituted by relatively preserved regions called frameworks (FR) joined by three hyper-variable regions called complementarity determining regions (CDR) (Kabat et al., 1991, Sequences of Proteins of Immunological Interest, 5^{th} Ed., NIH Publication No. 91-3242, Bethesda, Md.; Chothia & Lesk, 1987, J Mol Biol 196:901-17). The term “complementarity determining region” or “CDR”, as used herein, refers to the region within an antibody that complements an antigen's shape. Thus, CDRs determine the protein's affinity and specificity for specific antigens. The CDRs of the two chains of each pair are aligned by the framework regions, acquiring the function of binding a specific epitope. Consequently, in the case of VH and VL domains both the heavy chain and the light chain are characterised by three CDRs, respectively CDRH1, CDRH2, CDRH3 and CDRL1, CDRL2, CDRL3.

A number of definitions of the CDRs are commonly in use. The Kabat definition is based on sequence variability and is the most commonly used (see http://www.bioinfo.org.uk/abs/). The ImMunoGeneTics information system (IMGT) (see http://www.imgt.org) can also be used. According to this system, a complementarity determining region (CDR-IMGT) is a loop region of a variable domain, delimited according to the IMGT unique numbering for V domain. There are three CDR-IMGT in a variable domain: CDR1-IMGT (loop BC), CDR2-IMGT (loop C′C″), and CDR3-IMGT (loop FG). Other definitions of the CDRs have also been developed, such as the Chothia, the AbM and the contact definitions (see http://www.imgt.org).

The antibody conjugate of the present invention may comprise a full-length antibody or an antigen-binding fragment thereof.

The antibody conjugate of the present invention may comprise a full-length antibody. The full length antibody may be an IgG, an IgM, an IgA, an IgD or an IgE. The full length antibody may be an IgG or an IgM.

The terms “antibody fragment, “antigen-binding fragment”, “functional fragment of an antibody”, and “antigen-binding portion” are used interchangeably herein and refer to one or more fragments or portions of an antibody that retain the ability to specifically bind to an antigen. The antibody fragment may comprise, for example, one or more CDRs, the variable region (or portions thereof), the constant region (or portions thereof), or combinations thereof. Examples of antibody fragments include, but are not limited to, a Fab fragment, a F(ab′)₂ fragment, an Fv fragment, a single chain Fv (scFv), a domain antibody (dAb or VH), a single domain antibody (sdAb), a VHH, a nanobody, a diabody, a triabody, a trimerbody, and a monobody.

The antibody conjugate of the invention may comprise an antigen-binding domain which is based on a non-immunoglobulin scaffold. These antibody-binding domains are also called antibody mimetics. Non-limiting examples of non-immunoglobulin antigen-binding domains include an affibody, a fibronectin artificial antibody scaffold, an anticalin, an affilin, a DARPin, a VNAR, an iBody, an affimer, a fynomeran, abdurin/nanoantibody, a centyrin, an alphabody, a nanofitin, and a D domain.

The antibody may be bifunctional.

The antibody may be non-human, such as murine, rat or camelid, chimeric, humanised or fully human. The antibody may be synthetic.

Different antibody formats and modifications used to extend half-life and/or enhance drug delivery that are currently known in the art, or that will be developed in the future, also form part of the present invention.

The antibody for use in the antibody conjugate of the present invention binds specifically TRBC. The capacity of the antigen binding domain to bind specifically to TRBC can be determined by a number of assays that are available in the art. Preferably, the binding specificity of the antigen-binding domain is determined by an in vitro binding assay, such as surface plasmon resonance (SPR), radioimmunoassay (MA), enzyme-linked immunosorbent assay (ELISA), and competitive ELISA; by immunofluorescent techniques such as immunohistochemistry (IHC), fluorescence microscopy, and flow cytometry; or by immunoprecipitation.

The antibody for use in the antibody conjugate of the present invention may bind either TRBC1 or TRBC2. Methods to determine whether the antibody is specific for TRBC1 or TRBC2 are described in WO2015/132598.

Antibodies or antibody fragments can deliver various payloads to target cells that express TRBC. The efficacy of antibody-drug conjugates (ADCs) depends not only on binding affinity and specificity to the antigen, but also on internalisation. Using TRBC-specific antibodies having different binding affinities, the present inventors have determined that the kinetic rate constants of the TRBC-specific antibody conjugates are the factors that determine their internalisation properties upon binding to the target cell. Surprisingly, the best internalisation is obtained using antibodies having a fast dissociation. Moreover, the present inventors were able to determine binding affinities that display optimal internalisation properties on T cells.

The term “internalisation” or “cellular uptake” are used indistinctly in the context of the present invention and, as used herein, refer to a process also known as receptor mediated endocytosis. Endocytosis is a cellular process by which molecules or substances are transported into the cell via cell membrane engulfment. After internalisation, the antibody conjugate molecules are transported to the lysosome where the payload is released. Therefore, the rate and extent of antibody conjugate internalisation is of crucial importance to its effectiveness.

Numerous techniques, including microscopy and flow cytometry, may be used to identify antibodies with desired cellular uptake rates. Immunofluorescence microscopy-based colocalisation with endosomal proteins is also employed to monitor cellular uptake. Unlike immunofluorescence microscopy, flow cytometry with fluorescently labelled antibody enables a more rapid and quantitative assessment of antibody internalisation, and potentially greater throughput. In a flow cytometry-based assay, a fluorescently labelled secondary antibody may be used to measure how much antibody remains surface-bound after an incubation period. Alternatively, cells may be treated with fluorescently labelled primary antibodies prior to cell surface fluorescence quenching with an anti-fluorophore antibody.

Given that an ADC requires endocytosis and subsequent acidification to be effective, pH-sensitive labels are very useful for following the internalization of an antibody conjugate. Non-limiting examples of pH sensitive dyes include fluorescein, which exhibits bright fluorescence that is quenched as the pH drops, and pHrodo dyes, which display very low fluorescence at neutral pH and exhibit increasing fluorescence as the pH becomes more acidic. Methods for labelling antibodies with fluorescent dyes are well-known in the art.

The term “affinity”, as used herein, refers to the strength of interaction between an antibody's antigen binding site and an epitope. Affinity is usually measured as the equilibrium dissociation constant (KD), which is a ratio of the dissociation rate constant (k_{d} or k_{off}) and association rate constant (kₐ or kₒₙ), i.e. k_{d}/kₐ or k_{off}/kₒₙ, between the antibody and the antigen. KD and affinity are inversely related. The term “association rate constant” or “on-rate” or “kₐ” or “kₒₙ”, as used herein, refers to a constant used to characterize how quickly the antibody binds to its target. The term “dissociation rate constant” or “off-rate” or “k_{d}” or “k_{off}”, as used herein, refers to a constant used to characterize how quickly the antibody binds to its target. KD is measured in M; kₐ is measured in M⁻¹ s⁻¹; and k_{d} is measured in s⁻¹.

The affinity of an antigen-binding domain for any given antigen may be quantified using any conventional method including, without limitation, labelled-dependent methods, such as direct and indirect ELISA and radioimmunoassay methods, as well as label-free methods which enable a direct detection and measurement of interactions in real-time, such as surface plasmon resonance (SPR) and bio-layer interference.

The KD and the kinetic rate constants of the antibody used in the antibody conjugate of the invention may be measured by SPR. The assay may be carried out using different parameters and using a variety of apparatuses that are commercially available. For example, the KD and the kinetic rate constants may be measured on a Biacore T200 instrument using HBSP1 as the running and dilution buffer (GE Healthcare BioSciences), at a flow rate of 30 ml/min at 25° C. Kinetic rate constants may be obtained by curve fitting according to a 1:1 Langmuir binding model.

The present inventors have determined that optimal internalisation properties are displayed when the antibody has a dissociation rate constant k_{d} in the range of 0.001 s⁻¹ to 0.3 s⁻¹. The antibody for use in the antibody conjugate of the invention may have a k_{d} in the range of 0.001 s⁻¹ to 0.25 s⁻¹. The antibody for use in the antibody conjugate of the invention may have a k_{d} in the range of 0.001 s⁻¹ to 0.2 s⁻¹. The antibody for use in the antibody conjugate of the invention may have a k_{d} in the range of 0.001 s⁻¹ to 0.15 s⁻¹. The antibody for use in the antibody conjugate of the invention may have a k_{d} in the range of 0.001 s⁻¹ to 0.1 s⁻¹. The antibody for use in the antibody conjugate of the invention may have a k_{d} in the range of 0.002 s⁻¹ to 0.3 s⁻¹. The antibody for use in the antibody conjugate of the invention may have a k_{d} in the range of 0.003 s⁻¹ to 0.3 s⁻¹. The antibody for use in the antibody conjugate of the invention may have a k_{d} in the range of 0.004 s⁻¹ to 0.3 s⁻¹. The antibody for use in the antibody conjugate of the invention may have a k_{d} in the range of 0.005 s⁻¹ to 0.3 s⁻¹. The antibody for use in the antibody conjugate of the invention may have a k_{d} in the range of 0.006 s⁻¹ to 0.3 s⁻¹. The antibody for use in the antibody conjugate of the invention may have a k_{d} in the range of 0.007 s⁻¹ to 0.3 s⁻¹. The antibody for use in the antibody conjugate of the invention may have a k_{d} in the range of 0.008 s⁻¹ to 0.3 s⁻¹. The antibody for use in the antibody conjugate of the invention may have a k_{d} in the range of 0.009 s⁻¹ to 0.3 s⁻¹. The antibody for use in the antibody conjugate of the invention may have a k_{d} in the range of 0.01 s⁻¹ to 0.3 s⁻¹. The antibody for use in the antibody conjugate of the invention may have a k_{d} in the range of 0.01 s⁻¹ to 0.2 s⁻¹. The antibody for use in the antibody conjugate of the invention may have a k_{d} in the range of 0.01 s⁻¹ to 0.15 s⁻¹. The antibody for use in the antibody conjugate of the invention may have a k_{d} in the range of 0.01 s⁻¹ to 0.1 s⁻¹. The antibody conjugate according to claim 1, wherein the antibody has a k_{d} in the range of 0.002 s⁻¹ to 0.1 s⁻¹. The antibody conjugate according to claim 1, wherein the antibody has a k_{d} in the range of 0.017 s⁻¹ to 0.083 s⁻¹.

The antibody for use in the antibody conjugate of the invention may further have an association rate constant (kₐ) in the range of 1×10² M⁻¹ to 1×10⁶M⁻¹. The antibody for use in the antibody conjugate of the invention may further have a kₐ in the range of 5×10² M⁻¹ to 5×10⁵ M⁻¹. The antibody for use in the antibody conjugate of the invention may further have a kₐ in the range of 1×10³ M⁻¹ to 5×10⁵ M⁻¹. The antibody for use in the antibody conjugate of the invention may further have a kₐ in the range of 5×10³ M⁻¹ to 5×10⁵ M⁻¹.

The antibody for use in the antibody conjugate of the invention may further have a kₐ in the range of 1×10⁴ M⁻¹ to 1×10⁵ M⁻¹. The antibody for use in the antibody conjugate of the invention may further have a kₐ in the range of 5×10⁴ M⁻¹ to 1×10⁵ M⁻¹.

The antibody for use in the antibody conjugate of the invention may further have an affinity constant (KD) in the range of 1×10⁻¹⁰ M to 1×10⁻⁵M. The antibody for use in the antibody conjugate of the invention may further have a KD in the range of 1×10⁻¹⁰ M to 5×10−6 M. The antibody for use in the antibody conjugate of the invention may further have a KD in the range of 5×10⁻¹⁰ M to 1×10⁻⁶ M. The antibody for use in the antibody conjugate of the invention may further have a KD in the range of 5×10⁻¹⁰ M to 5×10⁻⁷ M. The antibody for use in the antibody conjugate of the invention may further have a KD in the range of 1×10⁻⁹ M to 5×10⁻⁷ M. The antibody for use in the antibody conjugate of the invention may further have a KD in the range of 5×10⁻⁹ M to 1×10⁻⁷ M.

The antibody for use in the antibody conjugate of the invention may have a k_{d} in the range of 0.001 s⁻¹ to 0.35 s⁻¹, a kₐ in the range of 1×10² M⁻¹ to 1×10⁶ M⁻¹ and a KD in the range of 1×10⁻¹° M to 1×10⁻⁵ M.

The antibody for use in the antibody conjugate of the invention may have an increased internalisation upon binding to a target cell compared to that of the reference antibody having a VH domain with the sequence shown in SEQ ID NO: 1 and a VL domain with the sequence shown in SEQ ID NO: 2.

The internalisation of the antibody conjugate of the invention upon binding to the target cell may be increased over that of the reference antibody. The internalisation of the antibody conjugate may be increased in at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 300%, at least 400%, at least 500%, at least 600%, at least 700%, at least 800%, at least 900%, at least 1,000%, at least 5,000%, or at least 10,000% over the internalisation of the reference antibody upon binding to the target cell. Methods to determine and quantitate the internalisation of an antibody conjugate have been described in detail previously.

The antibody for use in the antibody conjugate of the invention may bind specifically to TRBC1.

The antibody for use in the antibody conjugate of the invention, which is specific for TRBC1, may comprise one of the following mutations or mutation combinations compared to a reference antibody having a VH domain with the sequence shown in SEQ ID NO: 1 and a VL domain with the sequence shown in SEQ ID NO: 2:
G106A in the VH domain;
Y32F in the VH domain;
G31S in the VH domain;
G26P and T28K in the VH domain;
Y102M in the VH domain;

The antibody for use in the antibody conjugate of the invention, which is specific for TRBC1, may consist of a VH domain with the sequence shown in SEQ ID NO: 1 and a VL domain with the sequence shown in SEQ ID NO: 2, and one of the following mutations or mutation combinations:
G106A in the VH domain;
Y32F in the VH domain;
G31S in the VH domain;
G26P and T28K in the VH domain;
Y102M in the VH domain;
T28K, Y32F, A100N, and N103L in the VH domain, and N35K in the VL domain;
T28K, Y32F and A100N in the VH domain, and N35K in the VL domain; or
T28K, Y32F, A100N, and A107S in the VH domain.

The antibody for use in the antibody conjugate of the invention, which is specific for TRBC1, may comprise the following mutation compared to a reference antibody having a VH domain with the sequence shown in SEQ ID NO: 1 and a VL domain with the sequence shown in SEQ ID NO: 2:
G106A in the VH domain.

The antibody for use in the antibody conjugate of the invention, which is specific for TRBC 1, may consist of a VH domain with the sequence shown in SEQ ID NO: 1 and a VL domain with the sequence shown in SEQ ID NO: 2, and the following mutation:
G106A in the VH domain.

These specific combined mutations have been shown to alter binding to TRBC1 in a manner which is useful to ADC therapeutic strategies (see Table 1).

TABLE 1
Binding kinetics of TRBC1-specific antibodies. Mutations or
mutation combinations are in comparison to a reference antibody
having a VH domain with the sequence shown in SEQ ID NO: 1
and a VL domain with the sequence shown in SEQ ID NO: 2.

| | | TRBC1 | | |
|---|---|---|---|---|
| Clone | Mutation | Ka (1/Ms) | Kd (1/s) | KD (M) |
| aTRBC1 | N/A | 2.38E+05 | 6.18E−04 | 2.57E−09 |
| Mut5 | VH Y32F | 1.07E+05 | 0.04574 | 4.26E−07 |
| Mut8 | VH G31S | 9.83E+04 | 7.56E−04 | 7.69E−09 |
| Mut9 | VH G26P, T28K | 8.58E+04 | 0.002477 | 2.89E−08 |
| Mut10 | VH Y102M | 1.06E+05 | 0.009507 | 9.00E−08 |
| Mut11 | VH G106A | 8.73E+04 | 0.0177 | 2.03E−07 |
| Mut12 | VH T28K, Y32F, | 8.68E+04 | 0.06131 | 7.07E−07 |
| | A100N, N103L. | | | |
| | VL N35K | | | |
| Mut13 | VH T28K, Y32F, | 1.074E+5 | 0.1079 | 1.004E−6 |
| | A100N. VL N35K | | | |
| Mut14 | VH T28K, Y32F, | 9.002E+4 | 0.3228 | 3.586E−6 |
| | A100N, A107S | | | |

The antibody for use in the antibody conjugate of the invention may bind specifically to TRBC2.

The antibody for use in the antibody conjugate of the invention, which is specific for TRBC2, may comprise one of the following mutation combinations compared to a reference antibody having a VH domain with the sequence shown in SEQ ID NO: 1 and a VL domain with the sequence shown in SEQ ID NO: 2:
T28K, Y32F, A100N, Y102L and N103M in the VH domain, and N35R in the VL domain;
T28K, Y32F and A100N in the VH domain; or
T28R, Y32F and A100N in the VH domain.

The antibody for use in the antibody conjugate of the invention, which is specific for TRBC2, may consist of a VH domain with the sequence shown in SEQ ID NO: 1 and a VL domain with the sequence shown in SEQ ID NO: 2, and one of the following mutation:
T28K, Y32F, A100N, Y102L and N103M in the VH domain, and N35R in the VL domain;
T28K, Y32F and A100N in the VH domain; or
T28R, Y32F and A100N in the VH domain.

These specific combined mutations have been shown to alter binding to TRBC2 in a manner which is useful to ADC therapeutic strategies (see Table 2).

TABLE 2
Binding kinetics of TRBC2-specific antibodies. Mutations or
mutation combinations are in comparison to a reference antibody
having a VH domain with the sequence shown in SEQ ID NO: 1
and a VL domain with the sequence shown in SEQ ID NO: 2.

| | | TRBC2 | | |
|---|---|---|---|---|
| Clone | Mutation | Ka (1/Ms) | Kd (1/s) | KD (M) |
| Mut3 (KFN) | VH T28K, Y32F, | 8.30E+4 | 0.0386 | 4.79E−7 |
| | A100N | | | |
| Mut4 (RFN) | VH T28R, Y32F, | 7.76E+04 | 0.045 | 5.815E−7 |
| | A100N | | | |
| Mut15 | VH T28K, Y32F, | 4.379E+4 | 0.08323 | 1.901E−6 |
| | A100N, Y102L, | | | |
| | N103M. VL N35R | | | |

The antibody for use in the antibody conjugate of the invention, which is specific for TRBC2, may comprise the following mutations compared to a reference antibody having a VH domain with the sequence shown in SEQ ID NO: 1 and a VL domain with the sequence shown in SEQ ID NO: 2:
T28K, Y32F, A100N, and N103L in the VH domain, and N35K in the VL domain.

The antibody for use in the antibody conjugate of the invention, which is specific for TRBC2, may consist of a VH domain with the sequence shown in SEQ ID NO: 1 and a VL domain with the sequence shown in SEQ ID NO: 2, and the following mutation:
T28K, Y32F, A100N, and N103L in the VH domain, and N35K in the VL domain.

As used herein, the term “reference antibody” refers to a humanised JOVI-1 antibody, i.e. hJOVI-1, which comprises a VH domain with the sequence shown in SEQ ID NO: 1 and a VL domain with the sequence shown in SEQ ID NO: 2. Murine JOVI-1 has been previously disclosed by Viney et al. (Viney et al., 1992, Hybridoma 11:701-13).

(VH domain of hJOVI-1):
SEQ ID NO: 1
QVQLVQSGAEVKKPGASVKVSCKASGYTFTGYVMHWVRQAPGQGLEWMGF
INPYNDDIQSNERFRGRVTMTRDTSISTAYMELSRLRSDDTAVYYCARGA
GYNFDGAYRFFDFWGQGTMVTVSS
(VL domain of hJOVI-1):
SEQ ID NO: 2
DIVMTQSPLSLPVTPGEPASISCRSSQRLVHSNGNTYLHWYLQKPGQSPR
LLIYRVSNRFPGVPDRFSGSGSGTDFTLKISRVEAEDVGVYYCSQSTHVP
YTFGQGTKLEIK

The antibody for use in the antibody conjugate of the invention may comprise a VH domain with the sequence shown in SEQ ID NO: 1 and a VL domain with the sequence shown in SEQ ID NO: 2.

Variants or mutants of the antibody for use in the antibody conjugate of the invention which maintain its specificity and internalisation properties also form part of the present invention. The term “variant” or “mutant”, as used herein, refers to a polypeptide differing from a specifically recited polypeptide, i.e. reference or parent polypeptide by amino acid insertions, deletions, and/or substitutions, created using, for example, recombinant DNA techniques or by de novo synthesis. Variant and mutant are used indistinctly in the context of the present invention.

2.2. Therapeutic Entity or Payload

The antibody conjugate of the invention comprises an antibody that is attached to a therapeutic entity or payload. For the purposes of investigating the internalisation properties of the antibody conjugate, the payload may be replaced by a detection entity.

The term “therapeutic entity” or payload”, as used herein, refers to any molecule that inhibits or prevents the function of cells and/or causes destruction of cells (cell death), and/or exerts anti proliferative effects. The payload may be a drug or a radionuclide.

The antibody in the antibody conjugate of the invention may be conjugated to a chemotherapeutic entity, a radionuclide or a detection entity.

The term “chemotherapeutic entity”, as used herein, refers to any molecule that inhibits or prevents the function of cells and/or causes cell death, and/or exerts anti proliferative effects.

The resulting conjugate is hereinafter referred to as “the antibody drug conjugate (ADC) of the invention”. The chemotherapeutic entity may be a cytotoxic drug or cytotoxin. A number of classes of cytotoxic agents are known in the art to have potential utility in antibody molecules and can be used in the ADC described herein, including, without limitation, a tubulin inhibitor, a DNA damaging agent, a topoisomerase I inhibitor, and an RNA polymerase II inhibitor.

To date, microtubules have five known binding sites: vinca alkaloid binding site, taxane binding site, colchicine binding site, maytansine binding site, and the laulimalide binding site. Microtubule/tubulin inhibitors can be classified into two major categories according to their mechanisms of action: agents promoting tubulin polymerization and stabilizing microtubule structures (agents binding to the taxane binding site and the laulimalide binding site), and agents inhibiting tubulin polymerization and destabilizing microtubule structures (agents binding to the vinca alkaloid binding site, maytansine binding site, colchicine binding site. Non-limiting examples of tubulin inhibitors binding to the vinca alkaloid binding site include vincristine, vinblastine, vinflunine, halichondrin B, eribulin mesylate, cryptophycins, and dolastatins, such as the auristatins MMAF, MMAE, PF-06390101, MMAD, auristatin E, auristatin W analogue, auristatin f-HPA, amberstatin 269, and AGD-0182. Non-limiting examples of tubulin inhibitors binding to the maytansine binding site include maytansine and maytansinoids, such as DM1 and DM4. Non-limiting examples of tubulin inhibitors binding to the colchicine binding site include colchicine, 2-methoxyestradiol, sulphonamides, and Aspergillus derivatives. Non-limiting examples of tubulin inhibitors binding to the taxane binding site include paclitaxel, docetaxel, cyclostreptin, eleutherobin, ABI-007, ixabepilone, patupilone, and BMS-310705. Non-limiting examples of tubulin inhibitors binding to the laulimalide binding site include laulimalide and peloruside A.

DNA damaging agents include, without limitation, calicheamicins (ozogamicin), such as calicheamicin γ1; pyrrolobenzodiazepines, such as PDB talirine and SG3249; duocarmycins, such as DUBA; campthothecin analogues, such as SN38 and DX-8951; anthracyclines; and doxorubicins (adriamycin). Also included are alkylating agents, nitrosoureas, ethylenimines/methylmelamine, alkyl sulfonates, antimetabolites, pyrimidine analogues, epipodophylotoxins, platinium coordination complexes such as cisplatin and carboplatin enzymes such as L-asparaginase.

Topoisomerase I inhibitors include, without limitation, irinotecan, topotecan, and camptothecin.

RNA polymerase II inhibitors include, without limitation, amatoxins, such as α-amanitin.

Cytotoxins suitable for use in ADCs are also described in, for example, International Patent Application Publication Nos. WO 2015/155345 and WO 2015/157592.

The chemotherapeutic entity may be a biological response modifier such as IFNα, IL-2, G-CSF and GM-CSF; anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogues and leuprolide; and non-steroidal antiandrogens such as flutamide.

The antibody in the antibody conjugate of the invention may be conjugated to a radionuclide. The resulting conjugate is hereinafter referred to as “the antibody radionuclide conjugate (ARC) of the invention”. Radioimmunoconjugates have a unique theranostic (i.e. therapy and diagnostic) potential. For diagnosis purposes, the antibody may be labelled with a radionuclide compatible with imaging procedures, such as single photon emission computed tomography or positron emission tomography (PET). For therapeutic purposes, the choice of the radionuclide largely depends on the size of the tumour to be treated, with high-energy β-emitters, such as ⁹⁰Y, being suitable for the therapy of larger tumours, and medium-energy β-emitters, such as ¹³¹I and ¹⁷⁷Lu, being more effective for the treatment of smaller tumours.

Radionuclides suitable for use in ARCs are well known in the art and other radionuclides are also contemplated in the present invention. One of the main attractive features of radioimmunotherapy is the crossfire or bystander effect, i.e., the ability to damage cells in close proximity to the site of antibody localisation. In most cases, antibody radiolabeling is accomplished either by iodination of tyrosines or by conjugation of metal chelators, such as diethylenetriaminepentaacetic acid (DTPA) or 1,4,7,10-tetraazacyclododecane-1,4,7,10-tetraacetic acid (DOTA), to the antibody molecule.

The detectable entity may be a fluorescent entity, for example a fluorescent peptide or dye or label. The term “fluorescent entity”, as used herein, refers to a moiety which, following excitation, emits light at a detectable wavelength. Examples of fluorescent entities include, but are not limited to, fluorescein isothiocyanate (FITC), phycoerythrin (PE), allophycocyanin (APC), green fluorescent protein (GFP), enhanced GFP, red fluorescent protein (RFP), blue fluorescent protein (BFP) and mCherry. Particularly advantageous are pH-sensitive dyes or labels for following the internalization of an antibody conjugate. Non-limiting examples of pH sensitive dyes include fluorescein, which exhibits bright fluorescence that is quenched as the pH drops, and pHrodo dyes, which display very low fluorescence at neutral pH and exhibit increasing fluorescence as the pH becomes more acidic.

An antibody conjugate of the invention conjugated to a detectable entity may be used to determine the TRBC of a malignant T cell.

The antibody conjugate of the invention may comprise at least one therapeutic or payload molecule (including detection entities) conjugated thereto. The antibody conjugate of the invention may comprise any suitable number of The antibody conjugate of the invention may comprise conjugated thereto, i.e. 1, 2, 3, 4, 5, 6, 7, 8, 9, 10, or more payload molecules, to achieve a desired therapeutic effect.

2.3. Conjugation Chemistry

The antibody conjugate of the invention comprises an antibody that is attached to a chemotherapeutic entity, radionuclide or detection entity via conjugation or chemical linkers.

“Traditional” chemical conjugation of payload to the antibody occurs through solvent-exposed lysine residues (via succinimide ester derivatization) or via interchain cysteine residues (maleimide chemistry). Alternatively, a variety of approaches have been developed to obtain site-specific conjugations where sites of attachment of the cytotoxic agent to the antibody are precisely defined. These technologies are as follows: (a) applying engineered cysteine, such as THIOMAB enabling site-specific conjugation on only the heavy chain of the antibody; (b) introducing unnatural amino acids through mutagenesis to proteins and antibodies, such as incorporating p-acetylphenylalanine or selenocysteine into an IgG1; (c) using enzymatic and chemoenzymatic methods to generate site-specific conjugations, such as using engineered glycotransferases, transglutaminases, transpeptidase sortase, or formyl glycine generating enzymes to form the site-specific functionalization of antibodies; and (d) using tubulin-tag labelling strategies.

Where the antibody is a whole or monoclonal antibody, the payload may be chemically conjugated to the side chain of an amino acid, such as a cysteine, at a specific Kabat position in the Fc region of the antibody. Positions on the Fc region that are suitable for site-specific conjugation are well known in the art. Alternatively, the payload may be conjugated to the antibody through a thiol-maleimide linkage at the hinge and heavy-light chains.

Chemical linkers must be stable enough in systemic circulation and be able to rapidly and efficiently release the cytotoxic agent upon internalization of the antibody conjugate within cancer cells. According to the drug release mechanism, linkers for antibody conjugates are generally categorised into cleavable linkers (acid-labile linkers, protease cleavable linkers, and disulfide linkers) and non-cleavable linkers (Table 3). The antibody conjugate with non-cleavable linker requires lysosomal degradation of the antibody for releasing the cytotoxic agent, while that with cleavable linker involves hydrolysis, enzymatic reaction, or reduction to selectively release the cytotoxic drug based on the physiological environment to which the antibody conjugate is exposed.

TABLE 3
Summary of commonly used linkers for the
development of antibody conjugates.

| Abbreviation | Type |
|---|---|
| MHH | Chemical labile (acid labile) linker |
| DSDM | Disulfide-containing reducible linker |
| Sulfo-SPDB | Disulfide-containing reducible linker |
| MC-VC-PABC | Enzymatically cleavable linker |
| SMCC | Non-cleavable bifunctional linker |
| Mal-PEG-NHS | Non-cleavable spacer linker |
| GBC | Enzyme-labile ß-Glucuronide linker |

Three major types of cleavable linkers are often used in ADCs: acid-cleavable linkers, protease-cleavable linkers, and disulfide linkers. Acid-cleavable linkers such as hydrazine linkers (AcBut) are designed to be stable at a neutral pH of circulation but can be hydrolysed in lysosomes with a low pH environment. Protease-cleavable linkers are also used to keep antibody conjugates intact in systemic circulation and allow easy release of the cytotoxic drugs from antibody conjugates by lysosomal enzymes within cancer cells. For example, valine-citrulline (Val-Cit) and phenylalanine-lysine (Phe-Lys) confer excellent stability and PK/PD profile to antibody conjugates. In addition, the antibody conjugates with disulfide linkers, such as N-succinimidyl-4-(2-pyridyldithio)pentanoate (SPP) and N-succinimidyl-4-(2-pyridyldithio) butanoate (SPDB), take advantage of reduced glutathione with high intracellular concentrations to release free drug inside the cell. Antibody conjugates with reducible disulfide linkers generate uncharged metabolites that can diffuse into neighbouring cells and elicit bystander killing, which is advantageous for killing heterogeneous tumours.

Non-limiting examples of non-cleavable include thioether linkers (N-succinimidyl-4-[N-maleimidomethyl]cyclohexane-1-carboxylate [MCC]).

Other linkers include hydrophilic linkers, such as β-glucuronide linker, Sulfo-SPDB, and Mal-PEG4-NHS.

3. Pharmaceutical Composition

In another aspect, the present invention also relates to a pharmaceutical composition containing the antibody conjugate of the invention, hereinafter “the pharmaceutical composition of the invention”.

The pharmaceutical composition may additionally comprise a pharmaceutically acceptable carrier, diluent, excipient, or adjuvant. The pharmaceutical composition may optionally comprise one or more further pharmaceutically active polypeptides and/or compounds. Such a formulation may, for example, be in a form suitable for intravenous infusion.

The term “antibody conjugate of the invention” has been described in detail in the context of previous aspects of the invention and its features and embodiments apply equally to this aspects of the invention.

Administration

The administration of the antibody conjugate of the invention can be accomplished using any of a variety of routes that make the active ingredient bioavailable. For example, the agent can be administered by oral and parenteral routes, intraperitoneally, intravenously, subcutaneously, transcutaneously, intramuscularly, via local delivery for example by catheter or stent.

Typically, a physician will determine the actual dosage which will be most suitable for an individual subject and it will vary with the age, weight and response of the particular patient. The dosage is such that it is sufficient to reduce or deplete the number of malignant clonal T-cells.

4. Method of Treatment

In another aspect, the present invention provides an antibody conjugate of the invention for use in medicine.

In another aspect, the present invention provides a method for treating a T-cell lymphoma or leukaemia in a subject, hereinafter “the method of treatment of the invention”, which comprises the step of administering an antibody conjugate of the invention to a subject in need thereof. The administration step may be in the form of a pharmaceutical composition as described above.

This aspect of the invention may be alternatively formulated as an antibody conjugate of the invention for use in the treatment of a T-cell lymphoma or leukaemia, hereinafter “the antibody conjugate for use of the invention”.

This aspect of the invention may be alternatively formulated as the use of an antibody conjugate of the invention in the manufacture of a medicament for treating a T-cell lymphoma or leukaemia.

The term “antibody conjugate of the invention” has been described in detail in the context of previous aspects of the invention and its features and embodiments apply equally to these aspects of the invention.

A method for treating a T-cell lymphoma and/or leukaemia relates to the therapeutic use of the antibody conjugate of the invention, which may be administered to a subject having an existing T-cell lymphoma and/or leukaemia in order to lessen, reduce or improve at least one symptom associated with the disease and/or to slow down, reduce or block the progression of the disease.

The method for preventing a T-cell lymphoma and/or leukaemia relates to the prophylactic use of the antibody conjugate of the invention. Herein such antibody conjugate may be administered to a subject who has not yet contracted the T-cell lymphoma and/or leukaemia and/or who is not showing any symptoms of the T-cell lymphoma and/or leukaemia to prevent or impair the cause of the disease or to reduce or prevent development of at least one symptom associated with the disease. The subject may have a predisposition for, or be thought to be at risk of developing, the T-cell lymphoma and/or leukaemia.

These therapeutic applications will comprise the administration of a therapeutically effective amount of the antibody conjugate of the present invention.

The treatment of a T cell lymphoma or leukaemia in a subject may comprise the step of administrating the antibody conjugate to the subject, to cause selective depletion of the malignant T-cells, together with normal T-cells expressing the same TRBC as the malignant T-cells, but not to cause depletion of normal T-cells expressing the TRBC not expressed by the malignant T-cells.

The term “subject” or “individual”, as used in the context of the present invention, refers to members of mammalian species, preferably a male or female human being of any age or race.

The method may also comprise the step of investigating the TCR beta constant region (TCRB) of a malignant T cell from the subject to determine whether it expresses TRBC1 or

TRBC2. This information will assist the health practitioner on the appropriate TRBC selectivity of the antibody conjugate to be administered.

The term “therapeutically effective amount”, as used herein, refers to the amount of the antibody conjugate of the present invention which is required to achieve an appreciable prevention, cure, delay, reduction of the severity of, or amelioration of one or more symptoms of a T-cell lymphoma and/or leukaemia.

The method of the present invention may be used to treat a T-cell. “Lymphoma” is used herein according to its standard meaning to refer to a cancer which typically develops in the lymph nodes, but may also affect the spleen, bone marrow, blood and other organs. Lymphoma typically presents as a solid tumour of lymphoid cells. The primary symptom associated with lymphoma is lymphadenopathy, although secondary (B) symptoms can include fever, night sweats, weight loss, loss of appetite, fatigue, respiratory distress and itching.

The method of the present invention may be used to treat a T-cell leukaemia. “Leukaemia” is used herein according to its standard meaning to refer to a cancer of the blood or bone marrow.

The following is an illustrative, non-exhaustive list of diseases which may be treated by the method of the present invention.

Peripheral T-Cell Lymphoma

Peripheral T-cell lymphomas are relatively uncommon lymphomas and account fewer than 10% of all non-Hodgkin lymphomas (NHL). However, they are associated with an aggressive clinical course and the causes and precise cellular origins of most T-cell lymphomas are still not well defined.

Lymphoma usually first presents as swelling in the neck, underarm or groin. Additional swelling may occur where other lymph nodes are located such as in the spleen. In general, enlarged lymph nodes can encroach on the space of blood vessels, nerves, or the stomach, leading to swollen arms and legs, to tingling and numbness, or to feelings of being full, respectively. Lymphoma symptoms also include nonspecific symptoms such as fever, chills, unexplained weight loss, night sweats, lethargy, and itching.

The WHO classification utilizes morphologic and immunophenotypic features in conjunction with clinical aspects and in some instances genetics to delineate a prognostically and therapeutically meaningful categorization for peripheral T-cell lymphomas (Swerdlow et al.; WHO classification of tumours of haematopoietic and lymphoid tissues. 4th ed.; Lyon: IARC Press; 2008). The anatomic localization of neoplastic T-cells parallels in part their proposed normal cellular counterparts and functions and as such T-cell lymphomas are associated with lymph nodes and peripheral blood. This approach allows for better understanding of some of the manifestations of the T-cell lymphomas, including their cellular distribution, some aspects of morphology and even associated clinical findings.

The most common of the T-cell lymphomas is peripheral T-cell lymphoma, not otherwise specified (PTCL-NOS) comprising 25% overall, followed by angioimmunoblastic T-cell lymphoma (AITL) (18.5%)

Peripheral T-Cell Lymphoma, not Otherwise Specified (PTCL-NOS)

PTCL-NOS comprises over 25% of all peripheral T-cell lymphomas and NK/T-cell lymphomas and is the most common subtype. It is determined by a diagnosis of exclusion, not corresponding to any of the specific mature T-cell lymphoma entities listed in the current WHO 2008. As such it is analogous to diffuse large B-cell lymphoma, not otherwise specified (DLBCL-NOS).

Most patients are adults with a median age of 60 and a male to female ratio 2:1. The majority of cases are nodal in origin, however, extranodal presentations occur in approximately 13% of patients and most commonly involve skin and gastrointestinal tract.

The cytologic spectrum is very broad, ranging from polymorphous to monomorphous. Three morphologically defined variants have been described, including lymphoepithelioid (Lennert) variant, T-zone variant and follicular variant. The lymphoepithelioid variant of PTCL contains abundant background epithelioid histiocytes and is commonly positive for CD8. It has been associated with a better prognosis. The follicular variant of PTCL-NOS is emerging as a potentially distinct clinicopathologic entity.

The majority of PTCL-NOS have a mature T-cell phenotype and most cases are CD4-positive. 75% of cases show variable loss of at least one pan T-cell marker (CD3, CD2, CD5 or CD7), with CD7 and CD5 being most often downregulated. CD30 and rarely CD15 can be expressed, with CD15 being an adverse prognostic feature. CD56 expression, although uncommon, also has negative prognostic impact. Additional adverse pathologic prognostic factors include a proliferation rate greater than 25% based on KI-67 expression, and presence of more than 70% transformed cells. Immunophenotypic analysis of these lymphomas has offered little insight into their biology.

Angioimmunoblastic T-Cell Lymphoma (AITL)

AITL is a systemic disease characterised by a polymorphous infiltrate involving lymph nodes, prominent high endothelial venules (HEV) and peri-vascular expansion of follicular dendritic cell (FDC) meshworks. AITL is considered as a de-novo T-cell lymphoma derived from αβ T-cells of follicular helper type (TFH), normally found in the germinal centres.

AITL is the second most common entity among peripheral T-cell lymphoma and NK/T-cell lymphomas, comprising about 18.5% of cases. It occurs in middle aged to elderly adults, with a median age of 65 years old, and an approximately equal incidence in males and females. Clinically, patients usually have advanced stage disease, with generalised lymphadenopathy, hepatosplenomegaly and prominent constitutional symptoms. Skin rash with associated pruritus is commonly present. There is often polyclonal hypergammaglobulinemia, associated with autoimmune phenomena.

Three different morphologic patterns are described in AITL. The early lesion of AITL (Pattern I) usually shows preserved architecture with characteristic hyperplastic follicles. The neoplastic proliferation is localised to the periphery of the follicles. In Pattern II the nodal architecture is partially effaced with retention of few regressed follicles. The subcapsular sinuses are preserved and even dilated. The paracortex contains arborizing HEV and there is a proliferation of FDC beyond the B-cell follicle. The neoplastic cells are small to medium in size, with minimal cytologic atypia. They often have clear to pale cytoplasm, and may show distincT-cell membranes. A polymorphous inflammatory background is usually evident.

Although AITL is a T-cell malignancy, there is a characteristic expansion of B-cells and plasma cells, which likely reflects the function of the neoplastic cells as TFH cells. Both EBV-positive and EBV-negative B-cells are present. Occasionally, the atypical B-cells may resemble Hodgkin/Reed-Sternberg-like cells morphologically and immunophenotypically, sometimes leading to a diagnostic confusion with that entity. The B-cell proliferation in AITL may be extensive and some patients develop secondary EBV-positive diffuse large B-cell lymphomas (DLBCL) or—more rarely—EBV-negative B-cell tumors, often with plasmacytic differentiation.

The neoplastic CD4-positive T-cells of AITL show strong expression of CD10 and CD279 (PD-1) and are positive for CXCL13. CXCL13 leads to an increased B-cell recruitment to lymph nodes via adherence to the HEV, B-cell activation, plasmacytic differentiation and expansion of the FDC meshworks, all contributing to the morphologic and clinical features of AITL. Intense PD-1-expression in the perifollicular tumor cells is particularly helpful in distinguishing AITL Pattern I from reactive follicular and paracortical hyperplasia.

The follicular variant of PTCL-NOS is another entity with a TFH phenotype. In contradistinction to AITL, it does not have prominent HEV or extra-follicular expansion of FDC meshworks. The neoplastic cells may form intrafollicular aggregates, mimicking B-cell follicular lymphoma, but also can have interfollicular growth pattern or involve expanded mantle zones. Clinically, the follicular variant of PTCL-NOS is distinct from AITL as patients more often present with early stage disease with partial lymph node involvement and may lack the constitutional symptoms associated with AITL.

Anaplastic Large Cell Lymphoma (ALCL) ALCL may be subdivided as ALCL-anaplastic lymphoma kinase (ALK)+ or ALCL-ALK−. ALCL-ALK+ is one of the best-defined entities within the peripheral T-cell lymphomas, with characteristic “hallmark cells” bearing horseshoe-shaped nuclei and expressing ALK and CD30. It accounts for about 7% of all peripheral T-cell and NK-cell lymphomas and is most common in the first three decades of life. Patients often present with lymphadenopathy, but the involvement of extranodal sites (skin, bone, soft tissues, lung, liver) and B symptoms is common.

ALCL, ALK+ shows a wide morphologic spectrum, with 5 different patterns described, but all variants contain some hallmark cells. Hallmark cells have eccentric horseshoe- or kidney-shaped nuclei, and a prominent perinuclear eosinophilic Golgi region. The tumour cells grow in a cohesive pattern with predilection for sinus involvement. Smaller tumour cells predominate in the small cell variant, and in the lymphohistiocytic variant abundant histiocytes mask the presence of tumour cells, many of which are small.

By definition, all cases show ALK and CD30 positivity, with expression usually weaker in the smaller tumour cells. There is often loss of pan-T-cell markers, with 75% of cases lacking surface expression of CD3.

ALK expression is a result of a characteristic recurrent genetic alteration consisting of a rearrangement of ALK gene on chromosome 2p23 to one of the many partner genes, resulting in an expression of chimeric protein. The most common partner gene, occurring in 75% of cases, is Nucleophosmin (NPM1) on chromosome 5q35, resulting in t(2;5)(p23;q35). The cellular distribution of ALK in different translocation variants may vary depending on the partner gene.

ALCL-ALK− is included as a provisional category in the 2008 WHO classification. It is defined as a CD30 positive T-cell lymphoma that is morphologically indistinguishable from ALCL-ALK+ with a cohesive growth pattern and presence of hallmark cells, but lacking ALK protein expression.

Patients are usually adults between the ages of 40 and 65, in contrast to ALCL-ALK+, which is more common in children and young adults. ALCL-ALK− can involve both lymph nodes and extranodal tissues, although the latter is seen less commonly than in ALCL-ALK+. Most cases of ALCL-ALK− demonstrate effacement of lymph node architecture by sheets of cohesive neoplastic cells with typical “hallmark” features. In contrast to the ALCL-ALK+, the small cell morphologic variant is not recognised.

Unlike its ALK+ counterpart, ALCL-ALK− shows a greater preservation of surface T-cell marker expression, while the expression of cytotoxic markers and epithelial membrane antigen (EMA) is less likely. Gene expression signatures and recurrent chromosomal imbalances are different in ALCL-ALK− and ALCL-ALK+, confirming that they are distinct entities at a molecular and genetic level.

ALCL-ALK− is clinically distinct from both ALCL-ALK+ and PTCL-NOS, with significant differences in prognosis among these three different entities. The κ year overall survival of ALCL-ALK− is reported as 49% which is not as good as that of ALCL-ALK+(at 70%), but at the same time it is significantly better than that of PTCL-NOS (32%).

Enteropathy-Associated T-Cell Lymphoma (EATL)

EATL is an aggressive neoplasm which thought to be derived from the intraepithelial T-cells of the intestine. Two morphologically, immunohistochemically and genetically distinct types of EATL are recognised in the 2008 WHO classification: Type I (representing the majority of EATL) and Type II (comprising 10-20% of cases).

Type I EATL is usually associated with overt or clinically silent gluten-sensitive enteropathy, and is more often seen in patients of Northern European extraction due to high prevalence of celiac disease in this population.

Most commonly, the lesions of EATL are found in the jejunum or ileum (90% of cases), with rare presentations in duodenum, colon, stomach, or areas outside of the gastrointestinal tract. The intestinal lesions are usually multifocal with mucosal ulceration. Clinical course of EATL is aggressive with most patients dying of disease or complications of disease within 1 year.

The cytological spectrum of EATL type I is broad, and some cases may contain anaplastic cells. There is a polymorphous inflammatory background, which may obscure the neoplastic component in some cases. The intestinal mucosa in regions adjacent to the tumour often shows features of celiac disease with blunting of the villi and increased numbers of intraepithelial lymphocytes (IEL), which may represent lesional precursor cells.

By immunohistochemistry, the neoplastic cells are often CD3+CD4−CD8−CD7+CD5−CD56−βF1+, and contain cytotoxic granule-associated proteins (TIA-1, granzyme B, perforin). CD30 is partially expressed in almost all cases. CD103, which is a mucosal homing receptor, can be expressed in EATL.

Type II EATL, also referred to as monomorphic CD56+ intestinal T-cell lymphoma, is defined as an intestinal tumour composed of small- to medium-sized monomorphic T-cells that express both CD8 and CD56. There is often a lateral spread of tumour within the mucosa, and absence of an inflammatory background. The majority of cases express the γδ TCR, however there are cases associated with the αβ TCR.

Type II EATL has a more world-wide distribution than Type I EATL and is often seen in

Asians or Hispanic populations, in whom celiac disease is rare. In individuals of European descent EATL, II represents about 20% of intestinal T-cell lymphomas, with a history of celiac disease in at least a subset of cases. The clinical course is aggressive.

Hepatosplenic T-Cell Lymphoma (HSTL)

HSTL is an aggressive systemic neoplasm generally derived from γδ cytotoxic T-cells of the innate immune system, however, it may also be derived from αβ T-cells in rare cases. It is one of the rarest T-cell lymphomas, and typically affects adolescents and young adults (median age, 35 years) with a strong male predominance.

Extranodal Nk/T-Cell Lymphoma Nasal Type

Extranodal NK/T-cell lymphoma, nasal type, is an aggressive disease, often with destructive midline lesions and necrosis. Most cases are of NK-cell derivation, but some cases are derived from cytotoxic T-cells. It is universally associated with Epstein-Barr Virus (EBV).

Cutaneous T-Cell Lymphoma

The method of the present invention may also be used to treat cutaneous T-cell lymphoma.

Cutaneous T-cell lymphoma (CTCL) is characterised by migration of malignant T-cells to the skin, which causes various lesions to appear. These lesions change shape as the disease progresses, typically beginning as what appears to be a rash and eventually forming plaques and tumours before metastasizing to other parts of the body.

Cutaneous T-cell lymphomas include those mentioned in the following illustrative, non-exhaustive list; mycosis fungoides, pagetoid reticulosis, Sézary syndrome, granulomatous slack skin, lymphomatoid papulosis, pityriasis lichenoides chronica, CD30+ cutaneous T-cell lymphoma, secondary cutaneous CD30+ large cell lymphoma, non-mycosis fungoides CD30− cutaneous large T-cell lymphoma, pleomorphic T-cell lymphoma, Lennert lymphoma, subcutaneous T-cell lymphoma and angiocentric lymphoma.

The signs and symptoms of CTCL vary depending on the specific disease, of which the two most common types are mycosis fungoides and Sézary syndrome. Classic mycosis fungoides is divided into three stages:
Patch (atrophic or nonatrophic): Nonspecific dermatitis, patches on lower trunk and buttocks; minimal/absent pruritus;
Plaque: Intensely pruritic plaques, lymphadenopathy; and
Tumor: Prone to ulceration

Sézary syndrome is defined by erythroderma and leukemia. Signs and symptoms include edematous skin, lymphadenopathy, palmar and/or plantar hyperkeratosis, alopecia, nail dystrophy, ectropion and hepatosplenomegaly.

Of all primary cutaneous lymphomas, 65% are of the T-cell type. The most common immunophenotype is CD4 positive. There is no common pathophysiology for these diseases, as the term cutaneous T-cell lymphoma encompasses a wide variety of disorders.

The primary etiologic mechanisms for the development of cutaneous T-cell lymphoma (ie, mycosis fungoides) have not been elucidated. Mycosis fungoides may be preceded by a T-cell-mediated chronic inflammatory skin disease, which may occasionally progress to a fatal lymphoma.

Primary Cutaneous ALCL (C-ALCL)

C-ALCL is often indistinguishable from ALC-ALK− by morphology. It is defined as a cutaneous tumour of large cells with anaplastic, pleomorphic or immunoblastic morphology with more than 75% of cells expressing CD30. Together with lymphomatoid papulosis (LyP), C-ALCL belongs to the spectrum of primary cutaneous CD30-positive T-cell lymphoproliferative disorders, which as a group comprise the second most common group of cutaneous T-cell lymphoproliferations after mycosis fungoides.

The immunohistochemical staining profile is quite similar to ALCL-ALK—, with a greater proportion of cases staining positive for cytotoxic markers. At least 75% of the tumour cells should be positive for CD30. CD15 may also be expressed, and when lymph node involvement occurs, the differential with classical Hodgkin lymphoma can be difficult. Rare cases of ALCL-ALK+ may present with localised cutaneous lesions, and may resemble C-ALCL.

T-Cell Acute Lymphoblastic Leukaemia

T-cell acute lymphoblastic leukaemia (T-ALL) accounts for about 15% and 25% of ALL in paediatric and adult cohorts respectively. Patients usually have high white blood cell counts and may present with organomegaly, particularly mediastinal enlargement and CNS involvement.

The method of the present invention may be used to treat T-ALL which is associated with a malignant T cell which expresses a TCR comprising a TRBC.

T-Cell Prolymphocytic Leukaemia

T-cell-prolymphocytic leukemia (T-PLL) is a mature T-cell leukaemia with aggressive behaviour and predilection for blood, bone marrow, lymph nodes, liver, spleen, and skin involvement. T-PLL primarily affects adults over the age of 30. Other names include T-cell chronic lymphocytic leukaemia, “knobby” type of T-cell leukaemia, and T-prolymphocytic leukaemia/T-cell lymphocytic leukaemia.

In the peripheral blood, T-PLL consists of medium-sized lymphocytes with single nucleoli and basophilic cytoplasm with occasional blebs or projections. The nuclei are usually round to oval in shape, with occasional patients having cells with a more irregular nuclear outline that is similar to the cerebriform nuclear shape seen in Sézary syndrome. A small cell variant comprises 20% of all T-PLL cases, and the Sézary cell-like (cerebriform) variant is seen in 5% of cases.

T-PLL has the immunophenotype of a mature (post-thymic) T-lymphocyte, and the neoplastic cells are typically positive for pan-T antigens CD2, CD3, and CD7 and negative for TdT and CD1a. The immunophenotype CD4+/CD8− is present in 60% of cases, the CD4+/CD8+ immunophenotype is present in 25%, and the CD4−/CD8+ immunophenotype is present in 15% of cases

The T-cell lymphoma or leukaemia to be treated or prevented may be selected from peripheral T-cell lymphoma, not otherwise specified (PTCL-NOS); angio-immunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, T cell prolymphocytic leukaemia and T-cell acute lymphoblastic leukaemia.

The method of treatment may comprise a step of administering an antibody conjugate of the invention. The skilled person will be able to determine by conventional methods the amount of the antibody conjugate of the invention that are able to exert a therapeutic effect on the patient.

In another aspect, the present invention relates to an antibody conjugate of the invention for use in a method for targeting the delivery of a chemotherapeutic entity, a radionuclide or a detection entity to a cell which expresses TRBC in a subject.

The term “antibody conjugate of the invention” has been described in detail in the context of previous aspects of the invention and its features and embodiments apply equally to this aspect of the invention.

5. Methods of Personalised Medicine

Since T-cell malignancies are clonal, all malignant cells all express either TRBC1 or TRBC2. The present inventors previously demonstrated that immunotherapies targeting either TRBC1 or TRBC2 offer the ability to treat T-cell lymphomas, while potentially providing an acceptable toxicity profile (Maciocia et al., 2017, Nat Med 23:1416-23; WO2015/132598). The present invention also provides methods for identifying subjects with a T-cell lymphoma or leukaemia eligible for treatment with an antibody conjugate of the invention comprising determining the percentage of TRBC1 positive or TRBC2 positive T-cells in a sample comprising T-cells from the subject.

Thus, in another aspect, the present invention relates to a method for selecting a suitable therapy to treat a subject suffering from T-cell lymphoma or leukaemia, hereinafter “the first method of personalised medicine of the invention”, which comprises:
i) determining whether a malignant T cell in a sample isolated from the subject expresses TRBC1 or TRBC2; and
ii) selecting an antibody conjugate for use according to the invention based on the TRBC1 or TRBC2 expression of said malignant T cell.

The terms “antibody conjugate of the invention” and “subject” have been described in detail in the context of previous aspects of the invention and their features and embodiments apply equally to this aspect of the invention.

In the first method of personalised medicine of the invention, the subject is eligible to receive a therapy based on a conjugated antibody for use according to the invention specific for TRBC1 if the percentage of TRBC1 positive T-cells in the sample is 70%, or 75%, or 80%, or 85%, or 90%, or 95%, or 96%, or 97%, or 98%, or 99%, or more. Likewise, the subject is eligible for said treatment with an antibody conjugate of the invention specific for TRBC2 if the percentage of TRBC2 positive T-cells in the sample is 70%, or 75%, or 80%, or 85%, or 90%, or 95%, or 96%, or 97%, or 98%, or 99%, or more.

In another aspect, the present invention relates to a method for selecting a subject suffering from T-cell lymphoma or leukaemia to receive a therapy comprising an antibody conjugate for use according to the invention, hereinafter “the second method of personalised medicine of the invention”, which comprises:
i) determining whether a malignant T cell in a sample isolated from the subject expresses TRBC1 or TRBC2; and
ii) selecting said subject to receive a therapy based on an antibody conjugate for use according to the invention based on the TRBC1 or TRBC2 expression of said malignant T cell.

The terms “antibody conjugate of the invention” and “subject” have been described in detail in the context of previous aspects of the invention and their features and embodiments apply equally to this aspect of the invention.

In the second method of personalised medicine of the invention, the subject is eligible to receive a therapy based on a conjugated antibody for use according to the invention specific for TRBC1 if the percentage of TRBC1 positive T-cells in the sample is 70%, or 75%, or 80%, or 85%, or 90%, or 95%, or 96%, or 97%, or 98%, or 99%, or more. Likewise, the subject is eligible for said treatment with an antibody conjugate of the invention specific for TRBC2 if the percentage of TRBC2 positive T-cells in the sample is 70%, or 75%, or 80%, or 85%, or 90%, or 95%, or 96%, or 97%, or 98%, or 99%, or more.

In the first and second methods of personalised medicine of the invention, a sample comprising T-cells, such as a biological sample, is obtained from the subject to be studied. The term “sample” or “biological sample”, as used herein, includes different types of biological fluids or sections of tissues of the affected organs which comprise T-cells. Illustrative, non-limiting examples of samples useful in the diagnostic method of the invention include different types of biological fluids comprising T-cells, such as blood, lymph and spinal fluids. These biological fluid samples can be obtained by any conventional method known the skilled person. Alternatively, said sample can also be a section of an affected organ tissue sample, for example from a lymph gland, spleen, tonsil, or thymus, which can be obtained by any conventional method, for example by means of biopsy or surgical resection as well as from frozen sections taken for histologic purposes.

The sample may be, or may be derived from, a blood sample.

The first step (i) of determining whether a malignant T cell in a sample isolated from the subject expresses TRBC1 or TRBC2 may be carried out using an anti-TRBC1 and/or an anti-TRBC2 antibody, such as the ones described in the first aspect of the invention. This first step may use an anti-CD3 antibody, such as OKT3, to determine the total number of T cells.

There are a number of immunological methods available for determining whether a malignant T cell expresses TRBC1 or TRBC2 which are conventional. Non-limiting examples include immunohistochemistry and flow cytometry.

The T-cell lymphoma or leukaemia may be selected from peripheral T-cell lymphoma, not otherwise specified (PTCL-NOS); angio-immunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, T cell prolymphocytic leukaemia and T-cell acute lymphoblastic leukaemia.

### 6. Other Aspects of the Invention

6.1. TRBC1-Specific Antibody

In a further aspect, the present invention provides an antibody that binds specifically to TRBC1, hereinafter “the TRBC1-specific antibody of the invention”, which comprises one of the following mutation combinations in the VH domain compared to a reference antibody having a VH domain with the sequence shown in SEQ ID NO: 1 and a VL domain with the sequence shown in SEQ ID NO: 2:
G106A in the VH domain;
Y102M in the VH domain;
G26P and T28K in the VH domain; and
G31S in the VH domain.

The antibody for use in the antibody conjugate of the invention, which is specific for TRBC2, may consist of a VH domain with the sequence shown in SEQ ID NO: 1 and a VL domain with the sequence shown in SEQ ID NO: 2, with the following mutations:
G106A in the VH domain;
Y102M in the VH domain;
G26P and T28K in the VH domain; and
G31S in the VH domain.

The term “antibody” has been described in detail in the context of the first aspect of the invention and its features and embodiments apply equally to this aspect of the invention.

The antibody of the invention may be an antibody fragment maintaining the ability to bind specifically to TRBC1. The antibody fragment may be an antigen-binding domain, such as a scFv or a Fab.

6.2. TRBC2-Specific Antibody

In a further aspect, the present invention provides an antibody that binds specifically to TRBC1, hereinafter “the TRBC2-specific antibody of the invention”, which comprises one of the following mutation in the VH domain compared to a reference antibody having a VH domain with the sequence shown in SEQ ID NO: 1 and a VL domain with the sequence shown in SEQ ID NO: 2:
T28R, Y32F and A100N in the VH domain.

The antibody for use in the antibody conjugate of the invention, which is specific for TRBC2, may consist of a VH domain with the sequence shown in SEQ ID NO: 1 and a VL domain with the sequence shown in SEQ ID NO: 2, with the following mutations:
T28R, Y32F and A100N in the VH domain.

The term “antibody” has been described in detail in the context of the first aspect of the invention and its features and embodiments apply equally to this aspect of the invention.

The antibody of the invention may be an antibody fragment maintaining the ability to bind specifically to TRBC1. The antibody fragment may be an antigen-binding domain, such as a scFv or a Fab.

6.2. Chimeric Antigen Receptor

In another aspect, the present invention provides a chimeric antigen receptor (CAR), hereinafter “the CAR of the invention”, comprising the TRBC1-specific antibody of the invention or the TRBC2-specific antibody of the invention, a spacer, a transmembrane domain and an endodomain.

The terms “TRBC1-specific antibody of the invention” and “TRBC2-specific antibody of the invention” have been described in detail in previous aspects and their features and embodiments apply equally to this aspect of the invention.

The term “chimeric antigen receptor” or “CAR” or “chimeric T cell receptor” or “artificial T cell receptors” or “chimeric immunoreceptors”, as used herein, refers to a chimeric type I trans-membrane protein which connects an extracellular antigen-recognising domain (binder) to an intracellular signalling domain (endodomain). The binder is typically a single-chain variable fragment (scFv) derived from a monoclonal antibody (mAb), but it can be based on other formats which comprise an antigen binding site. A spacer domain is usually necessary to separate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 4-1BB which transmit survival signals.

Even more potent third generation CARs have now been described which have endodomains capable of transmitting activation, proliferation and survival signals.

When the CAR binds the target-antigen, this results in the transmission of an activating signal to the T-cell it is expressed on. Thus the CAR directs the specificity and cytotoxicity of the T cell towards tumour cells expressing the targeted antigen.

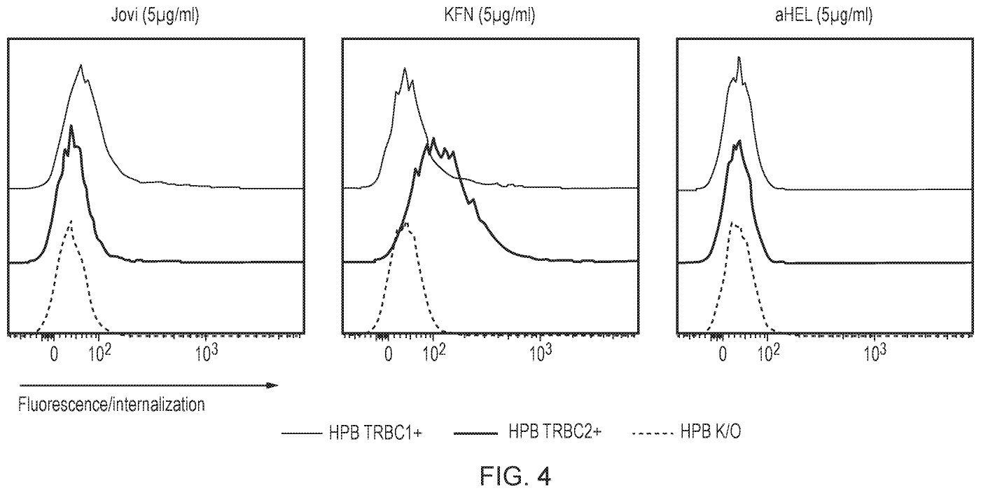

CARs typically therefore comprise: (i) an antigen-binding domain; (ii) a spacer; (iii) a transmembrane domain; and (iii) an intracellular domain which comprises or associates with a signalling domain (see FIG. 4).

A CAR may have the general structure:
Antigen-binding domain-spacer domain-transmembrane domain-intracellular signalling domain (endodomain).

The CAR of the present invention may comprise a signal peptide so that when the CAR is expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognised and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the SEQ ID NO: 3 to 5 or a variant thereof having 5, 4, 3, 2 or 1 amino acid mutations (insertions, substitutions or additions) provided that the signal peptide still functions to cause cell surface expression of the protein.

SEQ ID NO: 3:
MGTSLLCWMALCLLGADHADG

The signal peptide of SEQ ID NO: 3 is compact and highly efficient. It is predicted to give about 95% cleavage after the terminal glycine, giving efficient removal by signal peptidase.

SEQ ID NO: 4:
MSLPVTALLLPLALLLHAARP

The signal peptide of SEQ ID NO: 4 is derived from IgG1.

SEQ ID NO: 5:
MAVPTQVLGLLLLWLTDARC

The signal peptide of SEQ ID NO: 5 is derived from CD8.

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

In the CAR of the present invention, the spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs. The spacer may comprise a coiled-coil domain, for example as described in WO2016/151315.

The CAR of the present invention may comprise a sequence selected from the sequences shown as SEQ ID NOs: 6 to 10 or a variant thereof having at least 80% sequence identity.

(hinge-CH2CH3 of human IgG1)
SEQ ID NO: 6
AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
(human CD8 stalk):
SEQ ID NO: 7
TTTPAPRPPTPAPTIASQPLSLRPEACRPAAGGAVHTRGLDFACDI
(human IgG1 hinge):
SEQ ID NO: 8
AEPKSPDKTHTCPPCPKDPK
(CD2 ectodomain)
SEQ ID NO: 9
KEITNALETWGALGQDINLDIPSFQMSDDIDDIKWEKTSDKKKIAQFRKE
KETFKEKDTYKLFKNGTLKIKHLKTDDQDIYKVSIYDTKGKNVLEKIFDL
KIQERVSKPKISWTCINTTLTCEVMNGTDPELNLYQDGKHLKLSQRVITH
KWTTSLSAKFKCTAGNKVSKESSVEPVSCP
EKGLD
(CD34 ectodomain)
SEQ ID NO: 10
SLDNNGTATPELPTQGTFSNVSTNVSYQETTTPSTLGSTSLHPVSQHGNE
ATTNITETTVKFTSTSVITSVYGNTNSSVQSQTSVISTVFTTPANVSTPE
TTLKPSLSPGNVSDLSTTSTSLATSPTKPYTSSSPILSDIKAEIKCSGIR
EVKLTQGICLEQNKTSSCAEFKKDRGEGLARVLCGEEQADADAGAQVCSL
LLAQSEVRPQCLLLVLANRTEISSKLQLMKKHQSDLKKLGILDFTEQDVA
SHQSYSQKT
(COMP)
SEQ ID NO: 19
DLGPQMLRELQETNAALQDVRELLRQQVREITFLKNTVMECDACG

It is possible to truncate the COMP coiled-coil domain at the N-terminus and retain surface expression. The coiled-coil COMP spacer may therefore comprise or consist of a truncated version of SEQ ID NO: 18, which is truncated at the N-terminus. The truncated COMP may comprise the 5 C-terminal amino acids of SEQ ID NO: 19, i.e. the sequence CDACG (SEQ ID NO: 20). The truncated COMP may comprise 5 to 44 amino acids, for example, at least 5, 10, 15, 20, 25, 30, 35 or 40 amino acids. The truncated COMP may correspond to the C-terminus of SEQ ID NO: 19. For example a truncated COMP comprising 20 amino acids may comprise the sequence QQVREITFLKNTVMECDACG (SEQ ID NO: 21). Truncated COMP may retain the cysteine residue(s) involved in multimerisation. Truncated COMP may retain the capacity to form multimers.

The transmembrane domain is the sequence of the CAR that spans the membrane.

A transmembrane domain may be any protein structure which is thermodynamically stable in a membrane. This is typically an alpha helix comprising of several hydrophobic residues.

The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, CD8a or TYRP-1, which give good receptor stability.

In an embodiment, the transmembrane domain is derived from CD8a.

SEQ ID NO: 11: CD8a transmembrane domain
IYIWAPLAGTCGVLLLSLVIT

In another embodiment, the transmembrane domain is derived from TYRP-1.

SEQ ID NO: 12: TYRP-1 transmembrane domain
IIAIAVVGALLLVALIFGTASYLI

The endodomain is the signal-transmission portion of the CAR. After antigen recognition, receptors cluster, native CD45 and CD148 are excluded from the synapse and a signal is transmitted to the cell. The most commonly used endodomain component is that of CD3ζ which contains 3 ITAMs. This transmits an activation signal to the T cell after antigen is bound. CD3ζ may not provide a fully competent activation signal and additional co-stimulatory signalling may be needed. Examples of co-stimulatory domains include the endodomains from CD28, OX40, 4-1BB, CD27, and ICOS, which can be used with CD3ζ to transmit a proliferative/survival signal.

In an embodiment, at least one co-stimulatory endodomain is used with CD3. In a particular embodiment, the co-stimulatory endodomain is selected from the group consisting of the endodomains from CD28, OX40, 4-1BB, CD27, and ICOS.

In another embodiment, at least two co-stimulatory endodomains are used with CD3. In a particular embodiment, the two co-stimulatory endodomain are selected from the group consisting of the endodomains from CD28, OX40, 4-1BB, CD27, and ICOS, in any combination and order. Particularly suitable combinations include the endodomains from CD28 and CD3ζ, the endodomains of OX40 and CD3ζ, the endodomains of 4-1BB and CD3ζ, the endodomains from CD28, OX40 and CD3ζ, and the endodomains from CD28, 4-1BB and CD3ζ.

The transmembrane and intracellular T-cell signalling domain (endodomain) of a CAR with an activating endodomain may comprise the sequence shown as SEQ ID NO: 13 to 18 or a variant thereof having at least 80% sequence identity.

comprising CD28 transmembrane domain and CD3ζ
endodomain
SEQ ID NO: 13
FWVLVVVGGVLACYSLLVTVAFIIFWVRRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPR
comprising CD28 transmembrane domain and
CD28 and CD3ζ endodomains
SEQ ID NO: 14
FWVLVVVGGVLACYSLLVTVAFIIFWVRSKRSRLLHSDYMNMTPRRPGPT
RKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRR
GKGHDGLYQGLSTATKDTYDALHMQALPPR
comprising CD28 transmembrane domain and CD28,
OX40 and CD3ζ endodomains
SEQ ID NO: 15
FWVLVVVGGVLACYSLLVTVAFIIFWVRSKRSRLLHSDYMNMTPRRPGPT
RKHYQPYAPPRDFAAYRSRDQRLPPDAHKPPGGGSFRTPIQEEQADAHST
LAKIRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTA
TKDTYDALHMQALPPR
comprising CD8a transmembrane domain and CD3ζ
endodomain
SEQ ID NO: 16
IYIWAPLAGTCGVLLLSLVITRVLYCKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
comprising CD8a transmembrane domain and 4-1BB
and CD3ζ endodomains
SEQ ID NO: 17
IYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPR
comprising TYRP-1 transmembrane domain and 4-1BB
and CD3ζ endodomains
SEQ ID NO: 18
IIAIAVVGALLLVALIFGTASYLIKRGRKKLLYIFKQPFMRPVQTTQEED
GCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPR

A variant sequence may have at least 80%, 85%, 90%, 95%, 98% or 99% sequence identity to SEQ ID NO: 13 to 18, provided that the sequence provides an effective transmembrane domain and an effective intracellular T cell signalling domain.

A wide variety of molecules have been developed which are based on the basic concept of having two antibody-like binding domains.

Bispecific T-cell engaging molecules are a class of bispecific antibody-type molecules that have been developed, primarily for the use as anti-cancer drugs. They direct a host's immune system, more specifically the T cells' cytotoxic activity, against a target cell, such as a cancer cell. In these molecules, one binding domain binds to a T cell via the CD3 receptor, and the other to a target cells such as a tumour cell (via a tumour specific molecule). Since the bispecific molecule binds both the target cell and the T cell, it brings the target cell into proximity with the T cell, so that the T cell can exert its effect, for example, a cytotoxic effect on a cancer cell. The formation of the T cell:bispecific Ab:cancer cell complex induces signalling in the T cell leading to, for example, the release of cytotoxic mediators. Ideally, the agent only induces the desired signalling in the presence of the target cell, leading to selective killing.

Bispecific T-cell engaging molecules have been developed in a number of different formats, but one of the most common is a fusion consisting of two tandemly arranged single-chain variable fragments (scFvs) of different antibodies. These are sometimes known as BiTEs (Bi-specific T-cell Engagers).

6.3. Bispecific T-Cell Engager

The present invention also contemplates a bi-specific molecule which selectively recognises TRBC1 and is capable of engaging and activating a T cell. For example, the molecule may be a BiTE.

Thus, in another aspect, the present invention provides a bispecific T-cell engager (BiTE), hereinafter “the BiTE of the invention”, comprising the TRBC1-specific antibody of the invention or the TRBC2-specific antibody of the invention, and a T-cell activation domain.

The terms “TRBC1-specific antibody of the invention” and “TRBC2-specific antibody of the invention” have been described in detail in previous aspects and their features and embodiments apply equally to this aspect of the invention.

The term “T-cell activation domain”, as used herein, refers to a second domain capable of activating a T cell. The T-cell activation domain may be a scFv that binds specifically to

CD3. Examples of anti-CD3 scFv that are suitable for the purposes of the present invention are well known in the art and include, without limitation, the scFv derived from OKT3.

The bispecific molecule may comprise a signal peptide to aid in its production. The signal peptide may cause the bi-specific molecule to be secreted by a host cell, such that the bi-specific molecule can be harvested from the host cell supernatant.

The signal peptide may be at the amino terminus of the molecule. The bi-specific molecule may have the general formula: Signal peptide—variant antigen-binding domain of the invention—T-cell activation domain.

The bi-specific molecule may comprise a spacer sequence to connect the variant antigen-binding domain of the invention and a T-cell activation domain and spatially separate the two domains.

The spacer sequence may, for example, comprise an IgG1 hinge or a CD8 stalk. The linker may alternatively comprise an alternative linker sequence which has similar length and/or domain spacing properties as an IgG1 hinge or a CD8 stalk.

6.4. Nucleic Acid

In another aspect, the present invention also provides a nucleic acid sequence encoding the TRBC1-specific antibody of the invention or the TRBC2-specific antibody of the invention.

In another aspect, the present invention also provides a nucleic acid sequence encoding CAR of the invention.

In another aspect, the present invention also provides a nucleic acid sequence encoding a BiTE of the invention.

The terms “TRBC1-specific antibody of the invention”, “TRBC2-specific antibody of the invention”, “CAR of the invention”, and “BiTE of the invention” have been described in detail in the context of previous aspects of the invention and their features and embodiments apply equally to these aspects of the invention.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

The nucleic acid sequences and constructs of the invention may contain alternative codons in regions of sequence encoding the same or similar amino acid sequences, in order to avoid homologous recombination.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

6.5. Vector

The present invention also provides a vector, or kit of vectors, which comprises one or more nucleic acids encoding a TRBC1-specific antibody of the invention, TRBC2-specific antibody of the invention, a CAR of the invention, or a BiTE of the invention. Such a vector may be used to introduce the nucleic acid sequence into a host cell so that it expresses a TRBC1-specific antibody of the invention, or TRBC2-specific antibody of the invention, or a CAR of the invention, or a BiTE of the invention.

The terms “TRBC1-specific antibody of the invention”, “TRBC2-specific antibody of the invention”, “CAR of the invention”, and “BiTE of the invention”, and the nucleic acids encoding them have been described in detail in the context of previous aspects of the invention and their features and embodiments apply equally to these aspects of the invention.

The vector may, for example, be a plasmid or a viral vector, such as a retroviral vector or a lentiviral vector, or a transposon-based vector or synthetic mRNA.

The vector may be capable of transfecting or transducing a cytolytic immune cell, such as a T cell or NK cell.

6.6. Cell

Another aspect of the present invention relates to a cell, which comprises a CAR of the invention.

The cell may comprise a nucleic acid or a vector of the present invention.

The terms “CAR of the invention”, “nucleic acid of the invention”, “vector of the invention” have been described in detail in the context of previous aspects of the invention and their features and embodiments apply equally to these aspects of the invention.

The cell may be a cytolytic immune cell, such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumour cells, and are also implicated in transplant rejection. CTLs express the CD8 at their surface. These cells recognize their targets by binding to antigen associated with MEW class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cell of the invention may be any of the cell types mentioned above. In an embodiment, the cell of the invention is a T cell. In another embodiment, the cell of the invention is an NK cell.

Cells according to this aspect of the invention may either be created ex vivo either from a patient's own peripheral blood (1^{st} party), or in the setting of a haematopoietic stem cell transplant from donor peripheral blood (2^{nd} party), or peripheral blood from an unconnected donor (3^{rd} party).

Alternatively, cells according to this aspect of the invention may be derived from ex vivo differentiation of inducible progenitor cells or embryonic progenitor cells to cytolytic cells. Alternatively, an immortalised cytolytic cell line, such as a T or NK cell, which retains its lytic function and could act as a therapeutic may be used.

In all these embodiments, CAR-expressing cells are generated by introducing DNA or RNA coding for the chimeric polypeptide by one of many means including transduction with a viral vector, transfection with DNA or RNA.

The cell of the invention may be an ex vivo cell from a subject. The cell may be from a peripheral blood mononuclear cell (PBMC) sample. The cell, in particular, a cytolytic cell such as a T or NK cell, may be activated and/or expanded prior to being transduced with nucleic acid encoding the molecules providing the CAR of the invention, for example by treatment with an anti-CD3 monoclonal antibody.

The cell of the invention may be made by a method which comprises a step of transducing or transfecting a cell with a vector of the invention which comprises a nucleic acid sequence encoding the CAR.

The method for making a cell of the invention may further comprise a step of isolating the cell from a cell-containing sample from a subject or from other sources listed above, prior to the transduction or transfection step. Where the cell is a cytolytic cell, the sample is a cytolytic cell-containing sample from the subject.

The term “subject” or “individual”, as used in the context of the present invention, refers to members of mammalian species, preferably a male or female human being of any age or race.

The cell of the invention may then be purified, for example, by selection on the basis of expression of the antigen-binding domain of the CAR.

6.7. Pharmaceutical Composition

The present invention also relates to a pharmaceutical composition containing a TRBC1-specific antibody of the invention, or a TRBC2-specific antibody of the invention, or a cell or a plurality of cells of the invention, or a BiTE of the invention.

The terms “TRBC1-specific antibody of the invention”, “TRBC2-specific antibody of the invention”, “CAR of the invention”, and “BiTE of the invention” have been described in detail in the context of previous aspects of the invention and their features and embodiments apply equally to these aspects of the invention.

The description and embodiments of the pharmaceutical composition of previous aspects of the invention apply equally to these aspects of the invention. The skilled person will immediately know which modifications may be needed to adapt the pharmaceutical composition of previous aspects of the invention to this aspect.

6.8. Method of Treatment

In another aspect, the present invention provides a TRBC1-specific antibody of the invention, or a TRBC2-specific antibody of the invention, or a cell or a plurality of cells of the invention, or a BiTE of the invention for use in medicine.

In another aspect, the present invention provides a method for treating a T-cell lymphoma or leukaemia in a subject, which comprises the step of administering a TRBC1-specific antibody of the invention, or a TRBC2-specific antibody of the invention, or a cell or a plurality of cells of the invention, or a BiTE of the invention to a subject in need thereof. The administration step may be in the form of a pharmaceutical composition as described above.

This aspect of the invention may be alternatively formulated as a TRBC1-specific antibody of the invention, or a TRBC2-specific antibody of the invention, or a cell or a plurality of cells of the invention, or a BiTE of the invention for use in the treatment of a T-cell lymphoma or leukaemia.

This aspect of the invention may be alternatively formulated as the use of a TRBC1-specific antibody of the invention, or a TRBC2-specific antibody of the invention, or a cell or a plurality of cells of the invention, or a BiTE of the invention in the manufacture of a medicament for treating a T-cell lymphoma or leukaemia.

The terms “TRBC1-specific antibody of the invention”, “TRBC2-specific antibody of the invention”, “CAR of the invention”, and “BiTE of the invention” have been described in detail in the context of previous aspects of the invention and their features and embodiments apply equally to these aspects of the invention.

A method for treating a T-cell lymphoma and/or leukaemia relates to the therapeutic use of the antibody conjugate of the invention, which may be administered to a subject having an existing T-cell lymphoma and/or leukaemia in order to lessen, reduce or improve at least one symptom associated with the disease and/or to slow down, reduce or block the progression of the disease.

The description and embodiments of the method of treatment of previous aspects of the invention apply equally to these aspects of the invention. The skilled person will immediately know which modifications may be needed to adapt the method of treatment of previous aspects of the invention to this aspect.

6.9. Diagnostic Agent

It has been previously determined that the proportion of T cells from healthy donors that are TRBC1⁺ versus TRBC2⁺ is 35% vs. 65%, i.e. the median percentage of total T cells expressing TRBC1 was 35% (range, 25-47%) (Maciocia et al., 2017, Nat Med 23:1416-23). Since the T-cell lymphomas or leukaemias are clonal cancers (Maciocia et al., 2017; as above), the deregulated proliferation of malignant T cells characteristic of a T-cell lymphoma or leukaemia will result in a proportion of either TRBC1⁺ or TRBC2⁺ T-cells (i.e. TRBC2⁻ or TRBC1⁻ T-cells) that is significantly skewed. Accordingly, by binding specifically to TRBC1 and thus being capable of discriminating between TRBC1 and TRBC2, the antibody of the invention constitute useful agents for the diagnosis of T-cell lymphomas or leukaemias.

Thus, in another aspect, the present invention provides a diagnostic agent, hereinafter “the diagnostic agent of the invention”, which comprises a TRBC1-specific antibody of the invention or a TRBC2-specific antibody of the invention.

The terms “TRBC1-specific antibody of the invention” and “TRBC2-specific antibody of the invention” have been described in detail in the context of previous aspects of the invention and their features and embodiments apply equally to this aspect of the invention.

The TRBC1-specific antibody of the invention or the TRBC2-specific antibody of the invention to be used in these assays can be labelled or unlabelled. The term “detectable label” or “labelling agent”, as used herein, refers to a molecular label which allows the detection, localisation and/or identification of the molecule to which it is attached, using suitable procedures and equipment for detection, for example by spectroscopic, photochemical, biochemical, immunochemical or chemical means. Labelling agents that are suitable for labelling the antibodies include radionuclides, enzymes, fluorophores, chemiluminescent reagents, enzyme substrates or cofactors, enzyme inhibitors, particles, dyes and derivatives, and the like. As the person skilled in the art will appreciate, variant antigen-binding domains and antibodies that are not labelled need to be detected with an additional reagent, for example, a secondary antibody that is labelled. This is particularly useful in order to increase the sensibility of the detection method, since it allows the signal to be amplified. There is a wide range of conventional assays that can be used in the present invention which use an antibody of the invention that is not labelled (primary antibody) and an antibody of the invention that is labelled (secondary antibody); these techniques include Western blotting or immunoblot, ELISA (Enzyme-Linked Immunosorbent Assay), RIA (Radioimmunoassay), competitive EIA (Competitive Enzyme Immunoassay), DAS-ELISA (Double Antibody Sandwich-ELISA), immunocytochemical and immunohistochemical techniques, flow cytometry or multiplex detection techniques based on using protein microspheres, biochips or microarrays which include the antibody of the invention. Other ways of detecting and quantifying TRBC1 using the variant antigen-binding domain or antibody of the invention include affinity chromatography techniques or ligand binding assays.

The diagnostic agent may be used for diagnosing a T-cell lymphoma or leukaemia.

The T-cell lymphoma or leukaemia may be selected from peripheral T-cell lymphoma, not otherwise specified (PTCL-NOS); angio-immunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, T cell prolymphocytic leukaemia and T-cell acute lymphoblastic leukaemia.

6.10. Method of Diagnosis

In another aspect, the present invention provides a method for diagnosing a T-cell lymphoma or leukaemia in a subject, which comprises the step of contacting a TRBC1-specific antibody of the invention or a TRBC2-specific antibody of the invention to a sample comprising T-cells from the subject.

The terms “TRBC1-specific antibody of the invention”, “TRBC2-specific antibody of the invention”, “subject”, and “sample” have been described in detail in the context of previous aspects of the invention and their features and embodiments apply equally to this aspect of the invention.

The diagnostic method of the invention may additionally comprise a step of determining the total number of TRBC1 positive T-cells in the sample. The diagnostic method of the invention may additionally comprise a step of determining the total number of TRBC2 positive T-cells in the sample. The diagnostic method of the invention may additionally comprise a step of determining the total number of TRBC1 positive T-cells and TRBC2 positive T-cells in the sample.

The diagnostic method of the invention may additionally comprise a step of determining the total number of T-cells in the sample. This step may use an anti-CD3 antibody, such as OKT3, to determine the total number of T cells.

The diagnostic method of the invention may further comprise a step of determining the percentage of TRBC1 positive T-cells in the sample.

According to the first step of the diagnostic method of the invention, the TRBC1-specific antibody of the invention is contacted with a sample from the subject under study under suitable conditions known by the person in the art.

The diagnostic method of the invention may further comprise a step of determining the percentage of TRBC2 positive T-cells in the sample.

According to the first step of the diagnostic method of the invention, the TRBC2-specific antibody of the invention is contacted with a sample from the subject under study under suitable conditions known by the person in the art.

The person skilled in the art can use a number of conventional methods to detect TRBC1 or TRBC2 in the sample, which are suitable for carrying out the second step of the diagnostic method of the invention. Particularly useful are immunological methods. Thus, the use of the diagnostic agent of the invention may be particularly useful to perform the diagnostic method according to the invention. The features and particular embodiments of the diagnostic agent of the invention have been previously defined and apply equally to the diagnostic method of the invention.

In the diagnostic method of the invention, a percentage of 70%, or 75%, or 80%, or 85%, or 90%, or 95%, or 96%, or 97%, or 98%, or 99%, or more TRBC1 positive T-cells in the sample may be indicative of the presence of a T-cell lymphoma or leukaemia.

As it will be understood by those skilled in the art, the prediction, although preferred to be, need not be correct for 100% of the subjects to be diagnosed or evaluated. The term, however, requires that a statistically significant portion of subjects can be identified as having an increased probability of having a given outcome. Whether the data obtained from a subject is statistically significant can be determined without further ado by the person skilled in the art using various well-known statistic evaluation tools, e.g., determination of confidence intervals, p-value determination, cross-validated classification rates and the like etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.01, or 0.005 or lower.

Additionally, the TRBC1-specific antibody of the invention and the TRBC2-specific antibody of the invention, in view of their ability to specifically bind TRBC1 positive T-cells and TRBC2 positive T-cells, respectively, can also be used for in vivo diagnosis a T-cell lymphoma or leukaemia. For example, they can be used in medical imaging, i.e. a set of techniques and processes used to create images of a body (or parts and function thereof), e.g., a human body, for clinical purposes, such as medical procedures seeking to reveal, diagnose or examine a disease.

To this end, the variant antigen-binding domain or antibody of the invention are labelled by suitable methods known in the art, and are provided as agents for diagnostic imaging methods, such as radioimmunodiagnostics, positron emission tomography (PET), endoscopy immunofluorescent methods, etc., for example by means of coupling and/or loading with appropriate molecules, for example radioactive isotopes or fluorescent dyes. The variant antigen-binding domain and antibody of the invention may be coupled to gamma-emitting isotopes and used in radioimmunoscintigraphy using gamma cameras or single-photon emission computed tomography. The variant antigen-binding domain and antibody of the invention may be coupled to positron emitters and used in PET. The variant antigen-binding domain and antibody of the invention may be coupled to fluorescent dyes, such as Cy3, Cy2, Cy5 or FITC, and used in endoscopy immunofluorescent methods. The variant antigen-binding domain and antibody of the invention that are modified as described are administered by any suitable route, for example, intravenously, at an appropriate dose for the individual and the location of TRBC1 positive T-cells is detected, determined or measured by processes well known in the art. The methods and technologies used herein, including diagnostic imaging, are known to the skilled artisan, who can also provide suitable dose formulations.

The T-cell lymphoma or leukaemia to be diagnosed may be selected from peripheral T-cell lymphoma, not otherwise specified (PTCL-NOS); angio-immunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, T cell prolymphocytic leukaemia and T-cell acute lymphoblastic leukaemia.

The sample may be, or may be derived from, a blood sample.

6.11. Methods of Personalised Medicine

In another aspect, the present invention provides a method for identifying subjects with a T-cell lymphoma or leukaemia eligible for treatment with a cell, a TRBC1-specific antibody, a TRBC2-specific antibody, or BiTE of the invention, comprising determining the percentage of TRBC1 positive T-cells and/or TRBC2 positive T-cells in a sample comprising T-cells from the subject.

Thus, in another aspect, the present invention relates to a method for selecting a suitable therapy to treat a subject suffering from T-cell lymphoma or leukaemia, hereinafter “the first method of personalised medicine of the invention”, which comprises:
i) determining whether a malignant T cell in a sample isolated from the subject expresses TRBC1 or TRBC2; and
ii) selecting a cell, a TRBC1-specific antibody, a TRBC2-specific antibody, or BiTE for use according to the invention based on the TRBC1 or TRBC2 expression of said malignant T cell.

In another aspect, the present invention relates to a method for selecting a subject suffering from T-cell lymphoma or leukaemia to receive a therapy comprising a cell, a TRBC1-specific antibody, a TRBC2-specific antibody, or BiTE for use according to the invention, hereinafter “the second method of personalised medicine of the invention”, which comprises:
i) determining whether a malignant T cell in a sample isolated from the subject expresses TRBC1 or TRBC2; and
ii) selecting said subject to receive a therapy based on a cell, a TRBC1-specific antibody, a TRBC2-specific antibody, or BiTE for use according to the invention based on the TRBC1 or TRBC2 expression of said malignant T cell.

The terms “cell of the invention”, “TRBC1-specific antibody of the invention”, “TRBC2-specific antibody of the invention”, “BiTE of the invention”, “subject”, and “sample comprising T-cells” have been described in detail in the context of previous aspects of the invention and their features and embodiments apply equally to this aspect of the invention.

The description and embodiments of the methods of personalised medicine of previous aspects of the invention apply equally to these aspects of the invention. The skilled person will immediately know which modifications may be needed to adapt the methods of personalised medicine of previous aspects of the invention to this aspect.

### EXAMPLES

### Example 1: Antibody Internalisation of hJOVI-1 and KFN Antibodies

Cell uptake of the anti-TRBC1 antibody hJOVI-1 and the anti-TRBC2 antibody KFN was tested on HPB-ALL cells that were engineered to express only TRBC1, only TRBC2, or which were the TRBC was knocked-out (KO). To this aim, the pH-sensitive fluorophore

Zenon pHrodo iFL red (Thermo Scientific), which has a 560/585 nm spectrum, was used to conjugate hJOVI-1 and KFN in IgG format following the manufacturer's recommendations. The irrelevant anti-HEL antibody was also conjugated for use as control. The conjugated antibodies were incubated with HPB-ALL TRBC1, HPB-ALL TRBC2 and HPB-ALL KO cells for 24 h at 37° C., 5% CO2. Test antibodies hJOVI-1, KFN, and anti-HEL were applied at 5 μg/ml in a 96-well plate with 5×10⁴ cells/well in 100 μl of RPMI 10% FBS. After 24 h, cells were washed, harvested and stained with 1:100 LIVE/DEAD™ Fixable Violet Dead Cell Stain (Thermofisher) for 10 min before being analysed by flow cytometry using a Fortessa flow cytometer (BD).

Results shown in FIG. 4 demonstrate that both anti-TRBC antibodies hJOVI-1 and KFN internalise well into the HPB-ALL cells. However the KFN antibody which binds to its target at the same epitope albeit with a lower affinity, showed improved internalization over higher affinity hJOVI-1. Specificity of hJOVI-1 and KFN was maintained as antibody conjugates since hJOVI-1 only internalised into HPB-ALL TRBC1 cells and KFN only internalised into HPB-ALL TRBC2 cells.

### Example 2: Affinity and Binding Kinetics of TRBC Antibodies

Surface plasmon resonance (SPR) was carried out on several of antibody variants of hJOVI-1 to ascertain affinity, association and dissociation rates. The sequence details of these variants Mut1 to Mut15 used in the Examples section of the present patent application are included in Tables 1 and 2. These anti-TRBC antibody variants have specificity for either TRBC1 or TRBC2.

SPR experiments were performed using a Biacore T200 instrument using HBSP1 as the running and dilution buffer (GE Healthcare BioSciences). BIAevaluation software version 2.0 (GE Healthcare) was used for data processing. For determination of the binding kinetics, mouse anti-human IgG (GE Healthcare) was covalently coupled to a CM5 Sensor Chip (GE Healthcare). Anti-TRBC1 or anti-TRBC2 antibodies were captured onto the flow cell, and various concentrations of interaction partner protein (TRBC1 or TRBC2) were injected over the flow cell at a flow rate of 30 ml/min at a temperature of 25° C. A double reference subtraction was performed using buffer alone. Kinetic rate constants were obtained by curve fitting according to a 1:1 Langmuir binding model.

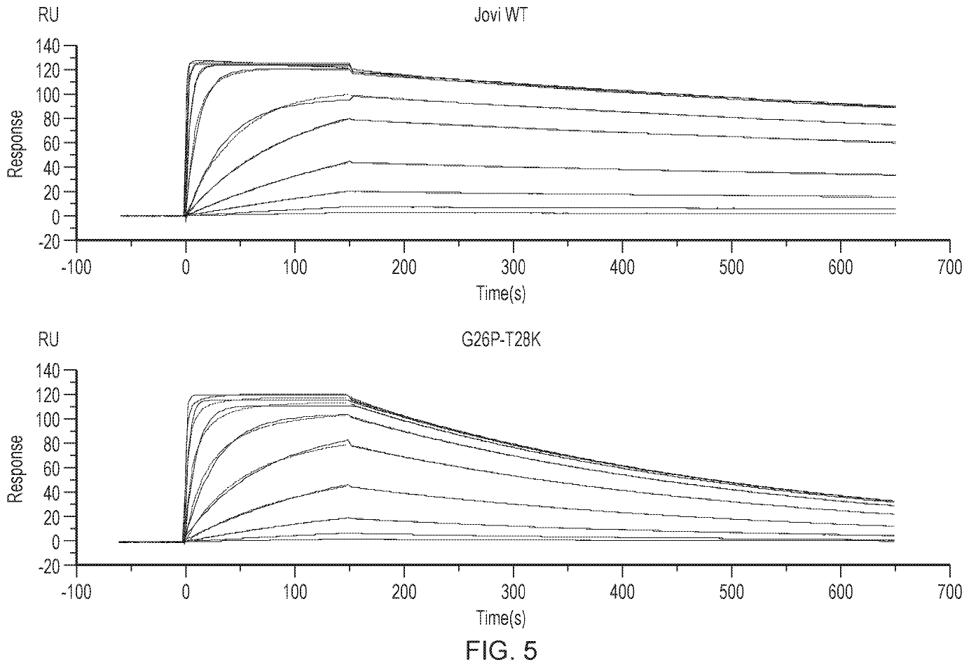

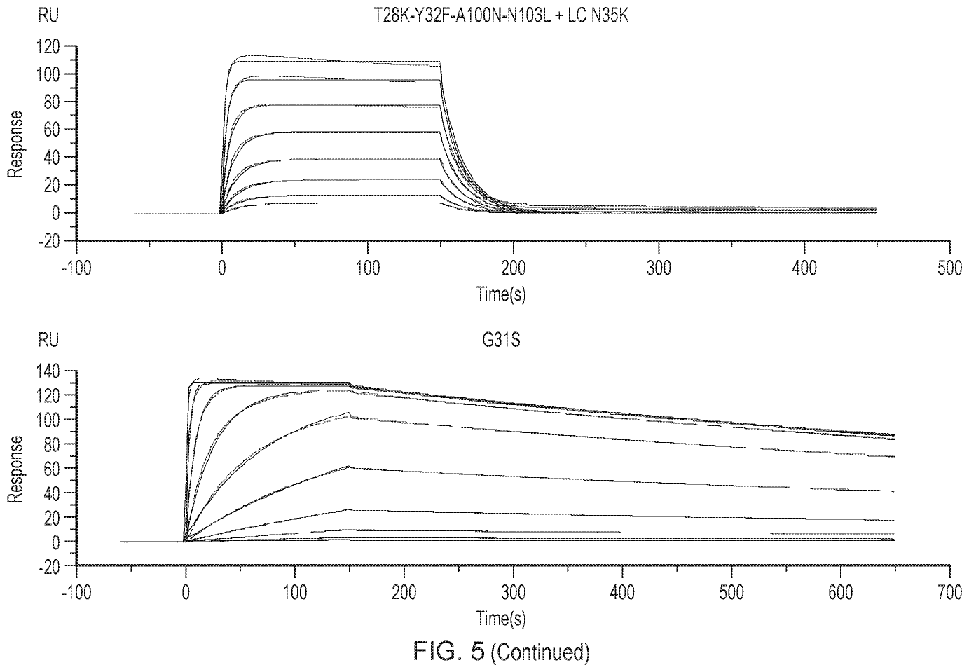

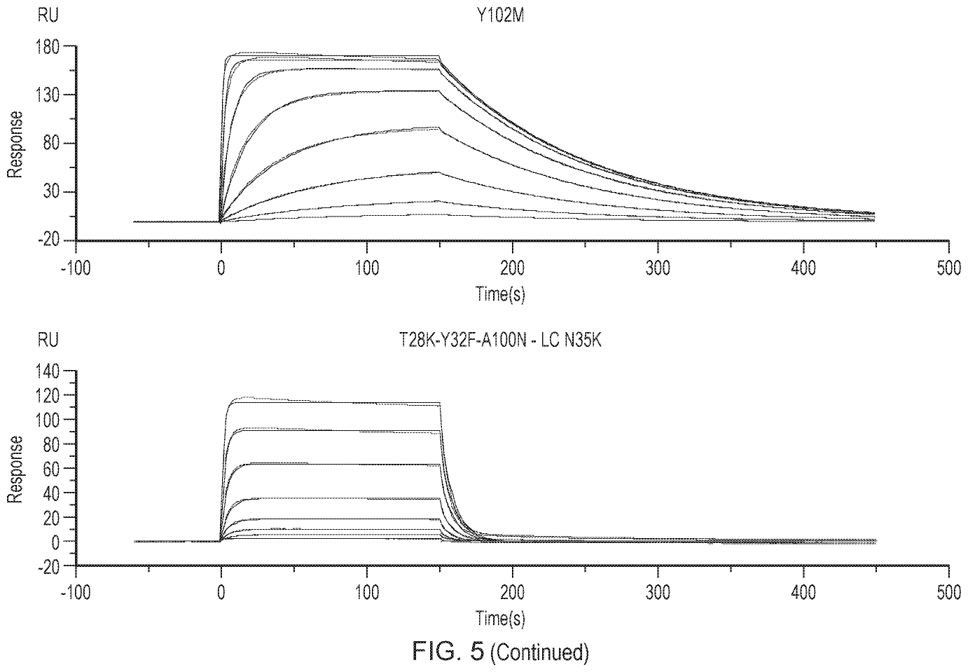

The affinity constant and kinetic rate constants obtained by SPR are shown in FIG. 5 and Table 1.

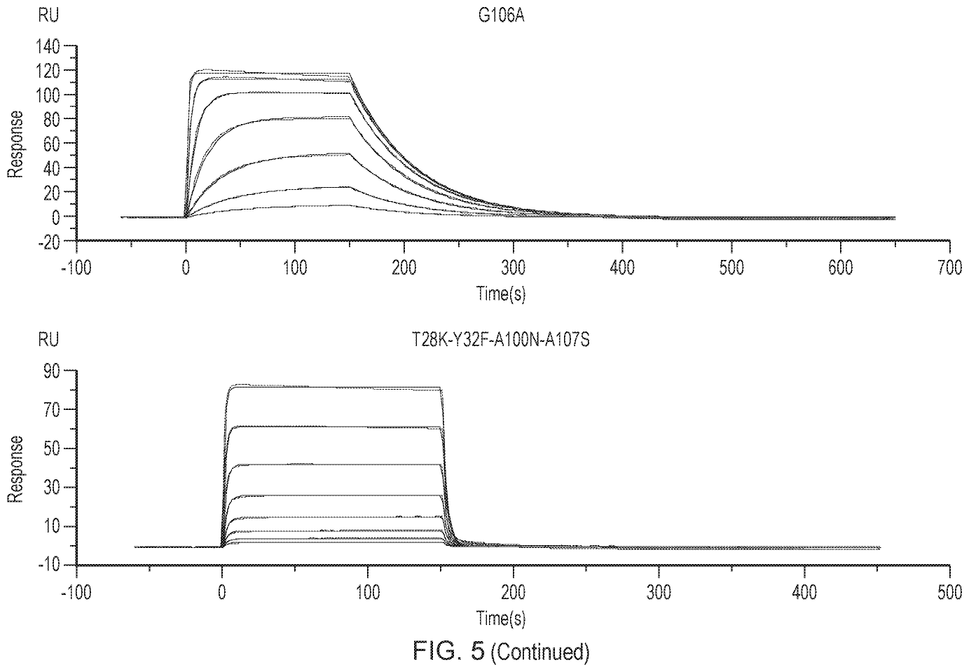

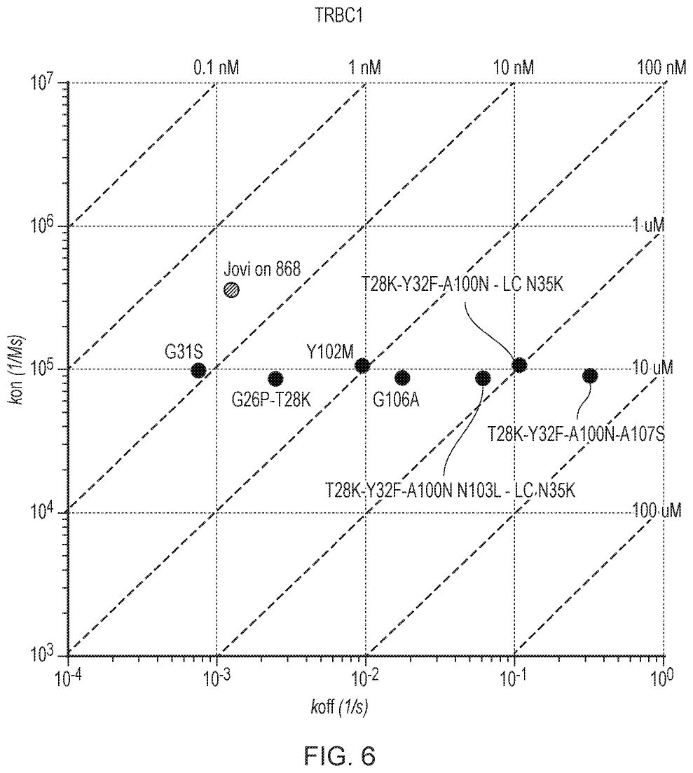

Various mutants with differing binding characteristics to TRBC1 were selected to compare their kinetic profiles on TRBC1 (FIG. 6). A number of binders with highly similar association rates but with different dissociation rates were identified and were further evaluated.

### Example 3: Antibody Internalisation of Anti-TRBC1 Antibodies

Internalisation of the anti-TRBC1 antibodies selected in Example 2 (FIG. 6) was assessed on HPB-ALL cells (TRBC1, TRBC2 and KO). To this aim, the pH-sensitive fluorophore Zenon pHrodo iFL green reagent (Thermo Scientific), which has a 509/533 nm spectrum, was used to conjugate the anti-TRBC1 antibodies in IgG format following the manufacturer's recommendations. The irrelevant anti-HEL antibody was also conjugated for use as control. The conjugated antibodies were incubated with HPB-ALL TRBC1, HPB-ALL TRBC2 and HPB-ALL KO cells for 9 h at 37° C., 5% CO2. Test antibodies were applied at 5 μg/ml in a 96-well plate with 5×10⁴ cells/well in 100 μl of RPMI 10% FBS. After 9 h, cells were washed, harvested and stained with 1:1000 fixable viability dye eFluor™ 780 (eBioscience) for 10 min before being analysed by flow cytometry using a Fortessa flow cytometer (BD).

At 9 hours, cells were washed with PBS, harvested at 400 g 5 mins and stained with 1:1000 fixable viability dye eFluor™ 780 (eBioscience™, Thermofisher) for 10 mins before being analysed by flow cytometry using a Fortessa flow cytometer (BD).

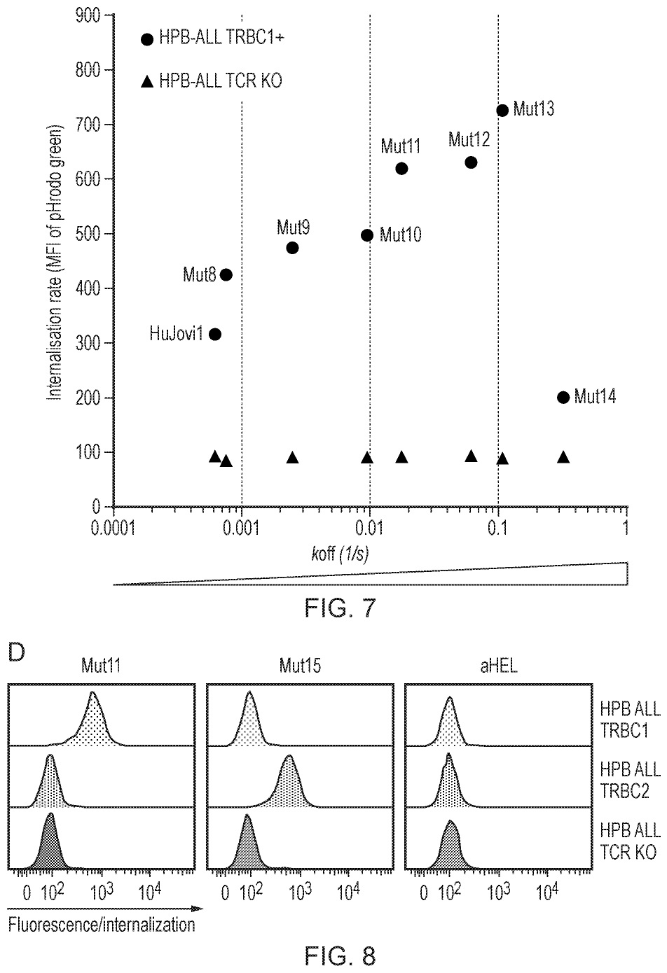

Results demonstrated that all anti-TRBC1 antibodies internalised well (FIG. 7). Analysis of the internalisation capacity of these variants showed that antibodies with decreasing affinities and increasing dissociation rates were more readily internalized. However, at a certain affinity (Mut14)—likely representing a lack of target antigen engagement—internalization was compromised. This is surprising because previous reports claimed that high affinities, and thus slow dissociation rates, are required to achieve optimal cell uptake.

### Example 4: Selection of Anti-TRBC1 and Anti-TRBC2 Antibodies for Optimal ADCs

Based on the findings of Example 3, i.e. that improved internalisation occurs at lower affinity, the anti-TRBC1 antibody Mut11 was selected for further investigation because it mimics the dissociation profile of the anti-TRBC2 clone KFN. Similarly, the TRBC2 antibody Mut15 was also selected because it also displays optimal binding kinetics.

Internalisation experiments using Mut11 and Mut15 were carried out as described in Example 3.

Results showed that both Mut11 and Mut15 were well internalised (FIG. 8). Moreover, the results demonstrated that the internalisation obtained with these two antibodies was much improved compared to that of hJovi1. The variant Mut11 showed the greatest internalisation on TRBC1 cells while maintaining antigen specificity. Similarly, a KFN variant Mut15 showed optimal internalisation and specificity against TRBC2 cells.

### Example 5: Generation of TRBC-Specific ADCs

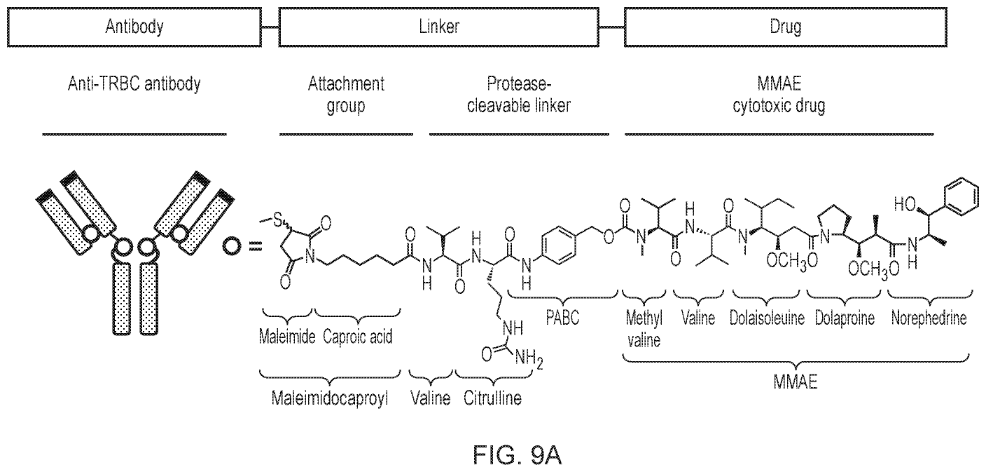

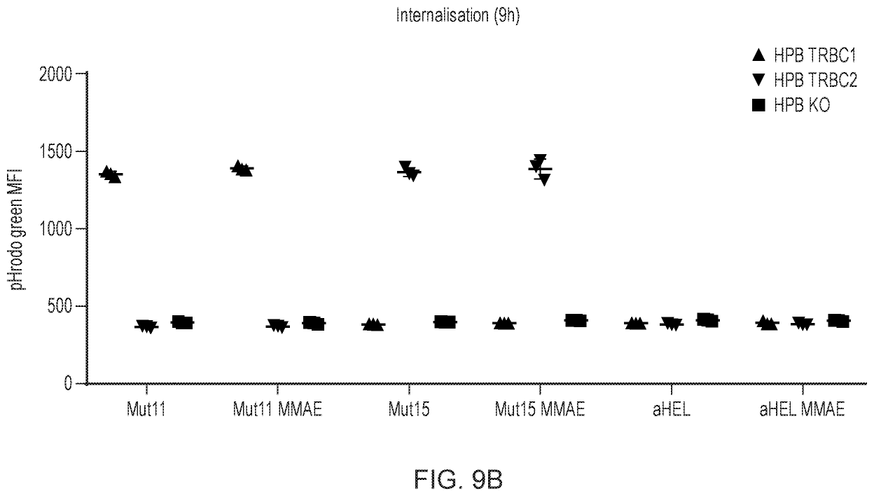

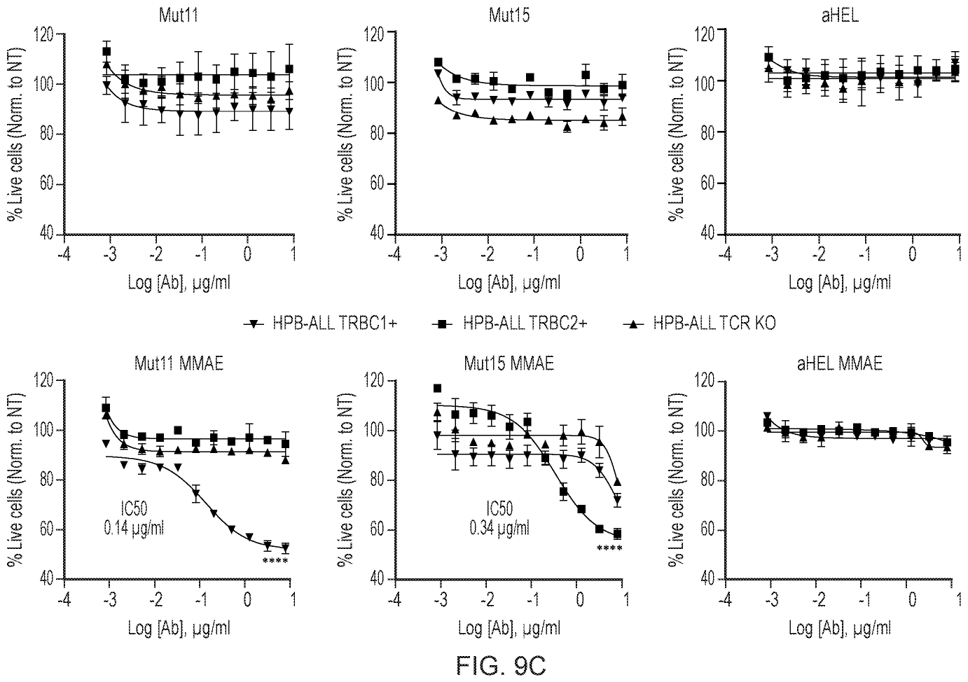

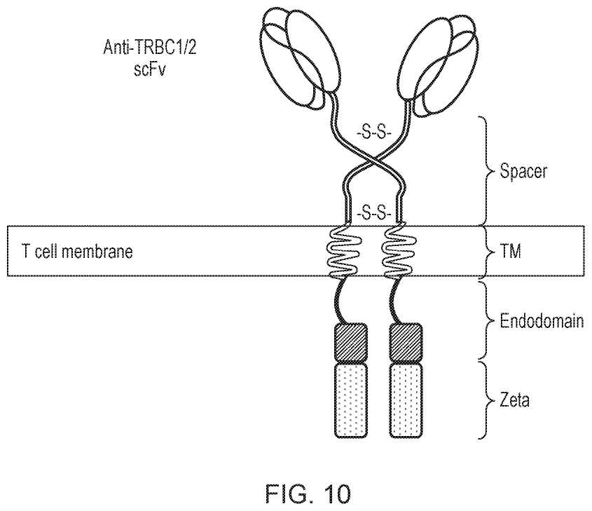

To test the efficacy of TRBC1 and TRBC2 ADC molecules, Mut11 and Mut15 antibodies were conjugated to monomethyl auristatin E (MMAE). A control anti-HEL was used. Briefly, antibodies were produced and purified to a high degree of purity. Antibody drug conjugates were generated using the linker payload mc-vc-PAB-MMAE (FIG. 9A). Briefly nucleophilic cysteine residues were first liberated from the reduced antibody inter-chain disulfide bonds using reducing agents. Reduced cysteines were conjugated with the drug-linker complex. Complexation conditions were optimised to identify ADCs with desired drug to antibody ratios (DAR). Drug-antibody ratio was assessed via hydrophobic interaction chromatography on a TSKgel Butyl-NPR column (2.5 μm, 4.6×100 mm) and assessed at 3.1, 3.2, and 3.3, for Mut11, Mut15, and anti-HEL, respectively.

Conjugation of Mut11 and Mut15 antibodies to monomethyl auristatin E (MMAE) did not impair ability for antibodies to be internalised (FIG. 9B).

### Example 6: Functional Characterisation of TRBC-Specific ADCs

In vitro cytotoxicity assays were performed on HPB-ALL TRBC1+, TRBC2+, and TCR KO at 5×10⁵ cells/ml stimulated with 10 ng/ml of human IL-7. ADC constructs were incubated with the target cells at a concentration of 8 μg/ml for 116 hours at 37° C. and then incubated with 10% culture volume of Alamar Blue for 4 hours. Supernatant was acquired on a plate reader (Varioscan Lux, Thermo Scientific) at 560/590 nm (excitation/emission) filter setting. Viability was expressed as normalisation with non-treated cells (treated×100/non-treated). IC50s were measured for each test compound along with non-conjugated control compounds to determine the concentration at which half maximal cell inhibition occurs.

To test the efficacy of TRBC1 and TRBC2 ADC molecules, MMAE-conjugated Mut11 and Mut15 antibodies were tested for their capacity to induce cell death. The half maximal inhibitory concentration for Mut11-MMAE and Mut15-MMAE to cause cell death against TRBC1-expressing and TRBC2-expressing cells were 0.14 and 0.34 μg/ml, respectively (FIG. 9C). These results demonstrate that anti-TRBC1 and anti-TRBC2 ADC molecules are effective at inducing internalisation, which results in cell death.

### Example 7: Generation of an Anti-TRBC1 CAR Based on hJOVI-1

Second generation CAR constructs (FIG. 4) are generated based on the following anti-TRBC1 antibodies, which comprises one of the following mutation combinations compared to a hJOVI1 (which has a VH domain with the sequence shown in SEQ ID NO: 1 and a VL domain with the sequence shown in SEQ ID NO: 2):
G106A in the VH domain;
Y102M in the VH domain;
G26P and T28K in the VH domain; and
G31S in the VH domain.

These CAR constructs are cloned into a retroviral vector and are used to transduce activated PBMCs obtained from healthy donors.

### Example 8: Functional Characterisation of an Anti-TRBC1 CAR: Cytokine Production

To assess the functional capacity of the anti-TRBC1 mutant CAR-T cells towards TRBC1, a plate bound assay is used in which TRBC1 or TRBC2 are immobilised prior to the addition of CAR-T cells. After 72 hours, culture supernatants are collected and IFN-γ production measured by ELISA.

### Example 9: Functional Characterisation of an Anti-TRBC1 CAR: Cytotoxicity Assay

To determine ability of the anti-TRBC1 triple mutant to target TRBC1, cytotoxicity assays are set up using Raji cells transduced to express either TRBC1 or TRBC2 as target cells and co-cultured with CAR-T cells. hJovi-1 CAR-T cells or anti-TRBC1 triple mutant CAR-T cells are cultured in a 1:1 (E:T) ratio with either Raji WT, Raji TRBC1⁺ or Raji TRBC2⁺ cells. Target cell recovery is measured 72 hours post culture by flow cytometry and used to establish cytotoxic capacity of CAR-T cells.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. An antibody conjugate that binds specifically to TCR beta constant region (TRBC), wherein the antibody has a dissociation rate constant (k_{d}) in the range of 0.001 s⁻¹ to 0.3 s⁻¹.

2. The antibody conjugate according to claim 1, wherein the antibody has a k_{d} in the range of 0.002 s⁻¹ to 0.1 s⁻¹.

3. The antibody conjugate according to any of claim 1 or 2, wherein the antibody is conjugated to a chemotherapeutic entity, a radionuclide or a detection entity.

4. The antibody conjugate according to claim 3, wherein the chemotherapeutic entity is a tubulin inhibitor.

5. The antibody conjugate according to claim 4, wherein the tubulin inhibitor is MMAE.

6. The antibody conjugate according to any of claims 1-5, wherein the antibody conjugate has an increased internalisation upon binding to a target cell compared to that of a reference antibody having a VH domain with the sequence shown in SEQ ID NO: 1 and a VL domain with the sequence shown in SEQ ID NO: 2.

7. The antibody conjugate according to any of claims 1 to 6, wherein the antibody binds specifically to TRBC1.

8. The antibody conjugate according to claim 7, wherein the antibody comprises one of the following mutations or mutation combinations compared to a reference antibody having a VH domain with the sequence shown in SEQ ID NO: 1 and a VL domain with the sequence shown in SEQ ID NO: 2:
G106A in the VH domain;
Y32F in the VH domain;
G31S in the VH domain;
G26P and T28K in the VH domain; or
Y102M in the VH domain.

9. The antibody conjugate according to claim 8, wherein the antibody comprises the following mutation compared to a reference antibody having a VH domain with the sequence shown in SEQ ID NO: 1 and a VL domain with the sequence shown in SEQ ID NO: 2:
G106A in the VH domain.

10. The antibody conjugate according to any of claims 1 to 6, wherein the antibody binds specifically to TRBC2.

11. The antibody conjugate according to claim 10, wherein the antibody comprises one of the following mutations or mutation combinations compared to a reference antibody having a VH domain with the sequence shown in SEQ ID NO: 1 and a VL domain with the sequence shown in SEQ ID NO: 2:
T28K, Y32F, A100N, Y102L and N103M in the VH domain, and N35R in the VL domain;
T28K, Y32F and A100N in the VH domain; or
T28R, Y32F and A100N in the VH domain.

12. The antibody conjugate according to claim 11, wherein the antibody comprises one of the following mutation combination compared to a reference antibody having a VH domain with the sequence shown in SEQ ID NO: 1 and a VL domain with the sequence shown in SEQ ID NO: 2:
T28K, Y32F, A100N, and N103L in the VH domain, and N35R in the VL domain.

13. An antibody conjugate according to any of claims 1 to 12 for use in the treatment of a T cell lymphoma or leukaemia.

14. The antibody conjugate for use according to claim 13, wherein the treatment of a T cell lymphoma or leukaemia in a subject comprises the step of administrating the antibody conjugate to the subject, to cause selective depletion of the malignant T-cells, together with normal T-cells expressing the same TRBC as the malignant T-cells, but not to cause depletion of normal T-cells expressing the TRBC not expressed by the malignant T-cells.

15. The antibody conjugate for use according to claim 14, wherein the method further comprises the step of investigating the TCR beta constant region (TCRB) of a malignant T cell from the subject to determine whether it expresses TRBC1 or TRBC2.

16. The antibody conjugate for use according to any of claims 13 to 15, wherein the T-cell lymphoma or leukaemia is selected from peripheral T-cell lymphoma, not otherwise specified (PTCL-NOS); angio-immunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma,primary cutaneous ALCL, T cell prolymphocytic leukaemia and T-cell acute lymphoblastic leukaemia.

17. An antibody conjugate according to any of claims 3 to 12 for use in a method for targeting the delivery of a chemotherapeutic drug to a cell which expresses TRBC in a subject.

18. A pharmaceutical composition which comprises an antibody conjugate according to any of claims 1 to 12 and a pharmaceutically acceptable carrier, diluent, excipient or adjuvant.

19. A method for selecting a suitable therapy to treat a subject suffering from T-cell lymphoma or leukaemia which comprises:
i) determining whether a malignant T cell in a sample isolated from the subject expresses TRBC1 or TRBC2; and
ii) selecting an antibody conjugate for use according to any of claims 13 to 16 based on the TRBC1 or TRBC2 expression of said malignant T cell.

20. A method for selecting a subject suffering from T-cell lymphoma or leukaemia to receive a therapy comprising an antibody conjugate for use according to any of claims 13 to 16, which comprises:
i) determining whether a malignant T cell in a sample isolated from the subject expresses TRBC1 or TRBC2; and
ii) selecting said subject to receive a therapy based on an antibody conjugate for use according to any of claims 13 to 16 based on the TRBC1 or TRBC2 expression of said malignant T cell.


SEQUENCE LISTING (21 sequences)

SEQ ID NO: 1 - protein, 124 aa, Artificial Sequence
  VH domain  (heavy chain variable domain) of humanised JOVI-1 antibody, hJOVI-1
QVQLVQSGAEVKKPGASVKVSCKASGYTFTGYVMHWVRQAPGQGLEWMGFINPYNDDIQSNERFRGRVTMTRDTSISTAYMELSRLRSDDTAVYYCARGAGYNFDGAYRFFDFWGQGTMVTVSS

SEQ ID NO: 2 - protein, 112 aa, Artificial Sequence
  VL domain  (light chain variable domain) of hJOVI-1
DIVMTQSPLSLPVTPGEPASISCRSSQRLVHSNGNTYLHWYLQKPGQSPRLLIYRVSNRFPGVPDRFSGSGSGTDFTLKISRVEAEDVGVYYCSQSTHVPYTFGQGTKLEIK

SEQ ID NO: 3 - protein, 21 aa, Artificial Sequence
  signal peptide
MGTSLLCWMALCLLGADHADG

SEQ ID NO: 4 - protein, 21 aa, Artificial Sequence
  signal peptide derived from IgG1
MSLPVTALLLPLALLLHAARP

SEQ ID NO: 5 - protein, 20 aa, Artificial Sequence
  signal peptide derived from CD8
MAVPTQVLGLLLLWLTDARC

SEQ ID NO: 6 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 7 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 8 - protein, 20 aa, Artificial Sequence
  spacer sequence, human IgG1 hinge
AEPKSPDKTHTCPPCPKDPK

SEQ ID NO: 9 - protein, 185 aa, Artificial Sequence
  spacer sequence, CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 10 - protein, 259 aa, Artificial Sequence
  spacer sequence, CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 11 - protein, 21 aa, Artificial Sequence
  CD8a transmembrane domain
IYIWAPLAGTCGVLLLSLVIT

SEQ ID NO: 12 - protein, 24 aa, Artificial Sequence
  TYRP-1 transmembrane domain
IIAIAVVGALLLVALIFGTASYLI

SEQ ID NO: 13 - protein, 140 aa, Artificial Sequence
  CD28 transmembrane domain and CD3 Zeta endodomain
FWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 180 aa, Artificial Sequence
  CD28 transmembrane domain and CD28 and CD3 Zeta endodomains
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 216 aa, Artificial Sequence
  CD28 transmembrane domain and CD28, OX40 and CD3 Zeta endodomains
FWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 136 aa, Artificial Sequence
  CD8a transmembrane domain and CD3 Zeta endodomain
IYIWAPLAGTCGVLLLSLVITRVLYCKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 178 aa, Artificial Sequence
  CD8a transmembrane domain and 4-1BB and CD3 Zeta endodomains
IYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 178 aa, Artificial Sequence
  TYRP-1 transmembrane domain and 4-1BB and CD3 Zeta endodomains
IIAIAVVGALLLVALIFGTASYLIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 45 aa, Artificial Sequence
  spacer sequence, coiled-coil COMP
DLGPQMLRELQETNAALQDVRELLRQQVREITFLKNTVMECDACG

SEQ ID NO: 20 - protein, 5 aa, Artificial Sequence
  spacer sequence, truncated coiled-coil COMP
CDACG

SEQ ID NO: 21 - protein, 20 aa, Artificial Sequence
  spacer sequence, truncated coiled-coil COMP (C-terminus)
QQVREITFLKNTVMECDACG